(19) United States
(12) Patent Application Publication
(10) Pub. No.: US 2024/0230664 A1
(43) Pub. Date: Jul. 11, 2024

(54) BEAD-BASED ASSAY FOR SIMULTANEOUS DETECTION OF BIOMOLECULES

(71) Applicants: CZ BIOHUB SF, LLC, San Francisco, CA (US); THE BOARD OF TRUSTEES OF THE LELAND STANFORD JUNIOR UNIVERSITY, Stanford, CA (US)

(72) Inventors: Xavier Rovira Clave, Stanford, CA (US); Alexandros Panagiotis Drainas, Stanford, CA (US); Sizun Jiang, Stanford, CA (US); Garry P. Nolan, Stanford, CA (US); Julien Sage, Stanford, CA (US)

(21) Appl. No.: 18/270,277

(22) PCT Filed: Jan. 7, 2022
(86) PCT No.: PCT/US22/11620

§ 371 Date: Jun. 29, 2023

Related U.S. Application Data
(60) Provisional application No. 63/199,571, filed on Jan. 8, 2021.

(51) Int. Cl. G01N 33/68; G01N 33/543

(52) U.S. Cl. CPC G01N 33/6854; G01N 33/54313; G01N 2469/20

(57) ABSTRACT

The present disclosure provides methods for determining the presence of at least one biomolecule in a sample. The present disclosure further provides methods for determining the presence of a coronavirus antibody in a sample.

### STATEMENT REGARDING FEDERALLY SPONSORED RESEARCH

This invention was made with Government support under contracts AI100627 and CA231997 awarded by the National Institutes of Health. The Government has certain rights in the invention.

### FIELD OF THE INVENTION

This invention is directed to methods for determining the presence of one or more biomolecule(s) in a sample. The methods described herein allows for simultaneous testing of multiple samples. The present disclosure further provides methods for determining the presence of coronavirus in a sample.

### BACKGROUND

Rapid, scalable and convenient detection of pathogens is critical for clinical purposes and population surveillance. Testing can be aimed at detecting current or past infection as extensively reviewed elsewhere (Esbin et al. 2020, RNA 26 (7): 771-83; Ravi et al. 2020, Biosensors & Bioelectronics 165 (112454): 112454). PCR-based testing is a highly sensitive method that reveals current infection, but it is slow, expensive, and requires trained personnel (Esbin et al. 2020, RNA 26 (7): 771-83). Antigen testing is more rapid and less expensive than PCR testing; however, utility of this type of test is limited by sensitivity and specificity issues (Winter and Hegde 2020, The Lancet Infectious Diseases. 20 (7); p755-874, e148-e179). Other alternatives for the detection of current infection include CRISPR- and LAMP-based testing, which are fast, easy, and sensitive (Ackerman et al. 2020, Nature, 582, 277-282; Lamb et al. 2020, medRxiv 19.20025155). Reliabilities of these methods as diagnostic tests have not been extensively studied yet. A common characteristic of all these types of tests is the inability to inform about past infections. Serology testing by lateral-flow assay reveals past infection and is rapid and inexpensive. Like antigen testing, the utility of serology testing by lateral-flow assay is hindered by sensitivity and specificity issues (Krammer and Simon 2020, Science (New York, N.Y.) 368 (6495): 1060-61). ELISA-based serology testing has an improved sensitivity and specificity compared to lateral-flow assays, but it is slow and limited in throughput (Krammer and Simon 2020, Science (New York, N.Y.) 368 (6495): 1060-61).

Most testing strategies offer only a binary readout and have difficulties with scalability to even the population level of towns, cities and counties. This means that these strategies are limited in scope if the problem escalates from a local to a global scale, as in the case with the COVID-19 pandemic. Robotic systems have been implemented to increase the throughput of PCR and ELISA, providing the ability to test samples from a few hundred patients simultaneously. However, the total number of tested patients is still far below of what is needed to control an active pandemic, limited by the low-throughput of these devices. Moving testing abilities from binary responses to more complex scenarios will allow assessment of crucial information, including testing for distinct molecular components in the same sample or detecting co-infection with other pathogens. State-of-the-art strategies that provide this type of multiplexing capability include CRISPR-based combinatorial arrayed reactions for multiplexed evaluation of nucleic acids (Ackerman et al. 2020, Nature, 582, 277-282) and protein microarrays for serological testing (Zhu et al. 2006, PNAS 03 (11): 4011-16; de Assis et al. 2020, BioRxiv). Unfortunately, neither of these strategies can achieve the scale at which population level testing would be possible.

Infectious disease outbreaks are a serious concern for public health. The pandemic of COVID-19 caused by severe acute respiratory syndrome coronavirus 2 (SARS-COV-2) is an illustrative example of how a localized outbreak can quickly escalate to a worldwide pandemic if not rapidly controlled. Among important interventions, extensive population testing to identify individuals experiencing current or past infection is key to controlling an infectious disease outbreak.

To monitor and control the spread of an outbreak, there is a need for new strategies to escalate testing capabilities to detect biomolecules and provide deeper understanding of the tested sample. While high-throughput techniques such as Rapid Extracellular Antigen Profiling (REAP) have been described previously (Wang et al., medRxiv. 2020; 20030593) such methods were limited to protein detection and yeast protein barcode libraries.

### SUMMARY OF THE INVENTION

In various aspects, the present disclosure provides methods for determining the presence of at least one biomolecule in a sample. In one embodiments, the method comprises: (a) obtaining a sample from a subject; (b) incubating the sample with a composition comprising a first bead, wherein said first bead is conjugated with (i) a first barcode moiety, (ii) a second barcode moiety, and (iii) a ligand capable of binding to the biomolecule, thereby producing a composition comprising a first construct, wherein said first construct comprises the first bead, the ligand and the biomolecule; (c) incubating the composition of (b) with a composition comprising a second bead, wherein said second bead is conjugated with an agent capable of binding to the biomolecule of the first construct, thereby producing a composition comprising a second construct, wherein said second construct comprises the first bead, the ligand, the biomolecule, the agent, and the second bead; (d) washing the composition of (c) under conditions that allow removal of unbound beads; (e) separating the first bead from the second bead under conditions that allow detection of the first bead comprising the first barcode moiety and the second barcode moiety; and (f) determining the presence of the first barcode moiety and the second barcode moiety.

In one embodiment, an aforementioned method is provided wherein the subject is an animal, plant, fungi, protist, bacterium or archaea. In various related embodiments, the animal includes but is not limited to, human, mouse or rat.

In still another embodiment, an aforementioned method is provided wherein the presence of 2, 3, 4, 5 or more biomolecules are determined.

In yet another embodiment, an aforementioned method is provided wherein the sample is a blood, serum, saliva, nasal/oropharyngeal swab, semen, urine, lymph, cerebrospinal fluid, interstitial fluid, spinal fluid, peritoneal fluid, pleural fluid, amniotic fluid, stool, bile, bone marrow, or skin sample.

In still another embodiment, an aforementioned method is provided wherein the first barcode moiety identifies the subject.

In still another embodiment, an aforementioned method is provided wherein the second barcode moiety identifies the ligand.

In yet another embodiment, an aforementioned method is provided wherein the biomolecule is an antibody, protein or a nucleic acid. In related embodiments the nucleic acid is an RNA or DNA. In related embodiments the RNA is RNA from a pathogen. In related embodiments the pathogen is a virus, bacterium, fungi, protozoa, or worm.

In yet another embodiment, an aforementioned method is provided wherein the ligand is an antigen or biomolecule-binding fragment of an antigen, biotinylated protein, polymer or a nucleic acid.

In still another embodiment, an aforementioned method is provided wherein the first bead is biotinylated.

In yet another embodiment, an aforementioned method is provided wherein the barcode moiety is an isotope, oligonucleotide or fluorophore barcode.

In still another embodiment, an aforementioned method is provided wherein the first bead is a polystyrene bead.

In yet another embodiment, an aforementioned method is provided wherein the second bead is magnetic.

In still another embodiment, an aforementioned method is provided wherein the agent is an antibody, protein or nucleic acid which binds to the biomolecule.

In yet another embodiment, an aforementioned method is provided wherein the separating step (e) comprises using specific proteolytic cleavages, chemical-based disruption, mechanical disruption, or heat-based dehybridization.

In still another embodiment, an aforementioned method is provided wherein the determining step (f) comprises using flow cytometry, mass spectrometry, fluorescence microscopy, electron microscopy, isotope-based imaging, Nuclear Magnetic Resonance (NMR), fluorescence resonance energy transfer (FRET), X-ray fluorescence (XRF), or nucleic acid sequencing.

In still another embodiment, an aforementioned method is provided wherein the incubating step (b) further comprises adding blocking agents to bind unbound antibody or nucleic acid on the first bead.

The present disclosure also provides, in one embodiment, a method for determining the presence of a coronavirus antibody in a sample comprising: (a) obtaining a blood sample from a human subject; (b) incubating the sample with a composition comprising a first bead, wherein said first bead is conjugated with (i) a first barcode moiety comprising a first isotope or a first combination of isotopes, (ii) a second barcode moiety comprising a second isotope or a second combination of isotopes, and (iii) a ligand comprising a coronavirus spike or nucleocapsid protein or fragment thereof capable of binding to the coronavirus antibody, thereby producing a composition comprising a first construct, wherein said first construct comprises the first bead, the ligand and the coronavirus antibody; (c) incubating the composition of (b) with a composition comprising a second bead, wherein said second bead is conjugated with an agent comprising an anti-human immunoglobulin antibody capable of binding to the coronavirus antibody of the first construct, thereby producing a composition comprising a second construct, wherein said second construct comprises the first bead, the ligand, the coronavirus antibody, the agent, and the second bead; (d) washing the composition of (c) under conditions that allow removal of unbound beads; (e) separating the first bead from the second bead under conditions that allow detection of the first bead comprising the first barcode moiety and the second barcode moiety; and (f) determining the presence of the first barcode moiety and the second barcode moiety.

In still another embodiment, an aforementioned method is provided wherein the coronavirus antibody is an anti-Spike protein antibody for SARS-COV-1 or SARS-COV-2.

In still another embodiment, an aforementioned method is provided wherein the barcode is an isotope- or oligonucleotide-based barcode. In related embodiments, the second barcode moiety identifies the ligand.

In yet another embodiment, an aforementioned method is provided wherein the determining step (f) comprises using mass cytometry, fluorescence microscopy, or nucleic acid sequencing.

In another embodiment, a method for determining the presence of at least one biomolecule in a sample. The method comprises (a) obtaining a sample from a subject; (b) incubating the sample with a composition comprising a plurality of ligand beads, wherein each ligand bead is conjugated with (i) a first barcode moiety that is unique for each sample, (ii) a second barcode moiety that is unique for each associated ligand, and (iii) a ligand capable of binding to the biomolecule, thereby producing a composition comprising a first construct, wherein said first construct comprises the first bead, the ligand and the biomolecule; (c) incubating the composition of (b) with a composition comprising a plurality of detection beads, wherein each detection bead is conjugated with an agent capable of binding to the biomolecule of the first construct, thereby producing a composition comprising a second construct, wherein said second construct comprises the first bead, the ligand, the biomolecule, the agent, and the second bead; (d) washing the composition of (c) under conditions that allow removal of unbound beads; (e) separating the first bead from the second bead under conditions that allow detection of the first bead comprising the first barcode moiety and the second barcode moicty; and (f) determining the presence of the first barcode moiety and the second barcode moicty.

In another embodiment, an aforementioned method is provided wherein the presence of a signal from the second barcode moiety is indicative of a current or prior infection from a pathogen associated with the ligand.

In various embodiments, an aforementioned method is provided wherein the agents capable of binding to the biomolecule of the first construct are antibodies.

In various embodiments, an aforementioned method is provided wherein the agents capable of binding to the biomolecule of the first construct are nucleic acids.

In various embodiments, an aforementioned method is provided wherein the antibodies comprise the same immunoglobulins capable of binding to one or more biomolecules.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIG. 1 shows the critical steps in diagnostic testing using the two-bead system.

FIGS. 2A-2B show the bead strategy for detection of antibodies against distinct proteins of a virus from samples from multiple individuals. (FIG. 2A). Polystyrene streptavidin beads are conjugated with biotinylated protein and polymers with chelated isotopes. Magnetic beads are coated with α-IgG. (FIG. 2B) Presence of an antibody in the patient's serum will bind the protein and will also be bound by the α-IgG connecting the two beads together.

FIG. 3 shows the Workflow used to identify antibodies against multiple viral targets in sera from previously infected or uninfected individuals using CyTOF as a readout. SN; supernatant. Patients' serum is added to plates. Samples are mixed with barcoded plates. The barcoded plates contain polystyrene beads with different combinations of elemental isotopes each chelated to a polymer (steps 1-2). The combinations are used to identify the protein sample (each well) and the protein bound (Spike S1, nucleoprotein N). The beads are then incubated (step 3) and excess antigens are added to block unbound antibodies. Samples are pooled, and magnetic beads are added to bind polystyrene beads that bound patient's antibodies (steps 6 and 7). The samples are washed using a magnet and the beads are then disassociated (steps 8-9). Samples are collected and analyzed by CyTOF.

FIG. 4 shows the two types of beads interact in the presence of a full-set of a pre-defined system of biomolecules. Streptavidin-coated polystyrene beads were coated with SARS-CoV-2 S1 biotinylated protein. Those beads were incubated in the presence or absence of a human anti-Spike antibody and washed. The complexes were incubated with anti-human IgG coated magnetic beads, stringently washed using a magnet and analyzed by fluorescence microscopy. Absence of anti-Spike (left) results in no binding of the two beads. Presence of anti-Spike (right) results in no binding of the two beads.

FIG. 5 shows a 6-fold dynamic range and a sensitivity on the femtogram range per μl. Streptavidin-coated polystyrene beads were coated with SARS-COV-2 S1 biotinylated protein. 10 μL reactions were generated with antibody concentrations ranging from 10 ng/μl to 100 fg/μL and a negative control. The reactions were then incubated with anti-human IgG coated magnetic beads and washed using a magnet. The beads were disassociated by low pH treatment and the Streptavidin-beads were collected and analyzed by flow cytometry. The dashed line indicates the control threshold. The presented assay demonstrates a sensitivity of 100 fg/μL. Several fold improvements are expected on the sensitivity and dynamic range of the assay.

FIGS. 6A and 6B show a two-bead assay identifies anti-SARS-COV-2 Spike S1 IgGs in COVID-19 convalescent plasma. (FIG. 6A) Number of Spike S1-loaded isotope-barcoded beads in the baseline, flowthrough, and release of twenty plasma samples obtained from COVID-19 infected individuals (“Convalescent”) and twenty plasma samples from before November 2019 (“Pre-pandemic”). (FIG. 6B) Correlation of the two-bead assay and standard ELISA. Rectangles indicate convalescent samples (labeled from c1 to c20). Dotted lines indicate the two-bead assay values for each of the twenty pre-pandemic samples. The two-bead assay value is the ratio between baseline-normalized release and flowthrough. Black dashed circles indicate two convalescent plasma samples (c9 and c10) with no detectable antibodies by the two-bead assay and ELISA.

FIGS. 7A-7C show anti-SARS-COV-2 Spike S1 IgG detection is achieved with less than 100 nL of plasma. Normalized flowthrough value (number of beads in flowthrough/number of beads in baseline) of 39 plasma samples obtained from COVID-19 infected individuals (“Convalescent”) and 55 plasma samples from donations occurred before November 2019 (“Pre-pandemic”). Reactions were run using (FIG. 7A) 100 nL, (FIG. 7B) 10 nL, and (FIG. 7C) 1 nL of plasma. Twelve replicates were run for each sample at each dilution, consisting of four target replicates (indicated by shades of gray) each one done in triplicate. The mean of the triplicate is shown.

FIG. 8 shows proof-of-concept of the simultaneous acquisition of 15360 serology tests. Normalized flowthrough value (number of beads in flowthrough/number of beads in baseline) of 39 plasma samples obtained from COVID-19 infected individuals (“Convalescent”) and 55 plasma samples from donations occurred before November 2019 (“Pre-pandemic”). Serology tests were run using 400 nL, 100 nL, 25 nL, and 6.25 nL of plasma. Beads were loaded with SARS-COV-2 Spike S1, Spike S1 RBD domain, Spike S1 NTD domain, Spike S2, Nucleocapsid, mutant Spike S1 RBD domain (alpha, gamma, delta, and kappa variants), and mutant Spike S1 (alpha, beta, delta, and B.1.617.3 variants). Two replicates were run for each sample at each dilution and target. The x-axis is organized from left to right: pre-pandemic samples and convalescent.

FIG. 9 shows a heatmap of anti-SARS-COV-2 Spike S1 IgG levels in 400 nL of COVID-19 convalescent and pre-pandemic plasma. Normalized flowthrough value (number of beads in flowthrough/number of beads in baseline) of 39 plasma samples obtained from COVID-19 infected individuals (“Convalescent”) and 55 plasma samples from donations occurred before November 2019 (“Pre-pandemic”). Serology tests were run using 400 nL of plasma. Beads were loaded with SARS-COV-2 Spike S1, Spike S1 RBD domain, Spike S1 NTD domain, Spike S2, Nucleocapsid, mutant Spike S1 RBD domain (alpha, gamma, delta, and kappa variants), and mutant Spike S1 (alpha, beta, delta, and B.1.617.3 variants). Two replicates, showed side-by-side, were run for each sample at each dilution and target.

FIGS. 10A-10D shows the presence of antibodies against distinct proteins do not always correlate. Correlations of normalized flowthrough value (number of beads in flowthrough/number of beads in baseline) of 39 plasma samples obtained from COVID-19 infected individuals (“Convalescent”) and 55 plasma samples from donations occurred before November 2019 (“Pre-pandemic”) for: (FIG. 10A) Nucleocapsid and Spike S2, (FIG. 10B) Nucleocapsid and Spike S1, (FIG. 10C) Spike S2 and Spike S1, and (FIG. 10D) Spike S1 alpha variant and Spike S1 beta variant. Serology tests were run using 400 nL (A to C), and 100 nL (D) of plasma. Two replicates were run for each sample.

FIGS. 11A-11D show schematics of testing for RNA from two different viral genes in samples from infected or uninfected individuals.

FIG. 12 shows schematics of the barcoding strategies for three distinct readouts.

FIG. 13 shows the generation of isotope-barcoded beads. Polymers were independently loaded with six metals and conjugated to biotin-SH. Three out of six biotinylated and isotope-loaded polymers were added on each well of a 96-well plate on a total of 20 wells (each well containing a distinct combination of isotopes). Conjugation buffer and streptavidin-coated beads were added to the well. After incubation, all beads were pooled, washed and analyzed by mass cytometry.

FIG. 14 shows mass cytometry analysis of Jurkat cells stained with isotopically labeled anti-CD45 following a 12-choose-6 strategy. Cells were stained with 12-choose-6 barcoded anti-CD45 and analyzed by mass cytometry. The left panel shows tSNE plots displaying groups of events with the dark shading indicating high isotope intensity and the light shading indicating low isotope intensity. Each group is positive for 6 of the 12 isotopes, as demonstrated on the right (Positive isotopes are labelled with a circle and negative isotopes are labelled with a square. All 924 possible combinations on the 12-choose-6 strategy were identified.

FIGS. 15A and 15B shows a proof-of-concept of Nanopore sequencing. (FIG. 15A) Number of reads across difference read lengths from Nanopore sequencing. The number on each peak indicate the number of concatenated DNA sequences. (FIG. 15B) Number of debarcoded oligo counts from Nanopore sequencing. Roughly ˜1 M oligo barcodes were detected from ˜350 k nanopore reads.

FIG. 16 shows a two-bead assay identifies anti-SARS-COV-2 Spike S1 IgGs in COVID-19 convalescent plasma using Nanopore sequencing. 16 samples, 8 positive and 8 negative samples were subjected to the two-bead assay. Prior mixing the samples, an aliquot was quantified using flow cytometry measuring the median intensity of Spike S1 with a fluorescent anti-IgG antibody (y-axis). Then the samples were mixed and processed using the nanopore sequencing pipeline. For each sample the number of barcodes is identified (x-axis). The Nanopore sequencing approach identified all positive samples (c1, c3, c12, c13, c16, c17, c19, c20) and two false positive samples (n2, n19). Dashed lines indicate the cut-off between positive and negative samples in each axis.

### DETAILED DESCRIPTION

The present disclosure addresses the aforementioned unmet need by providing methods and materials for determining the presence of one or more biomolecule(s) in a sample. In various aspects, the biomolecule is an antibody, protein or a nucleic acid.

As described herein, various embodiments the present disclosure provides simultaneous detection of multiple pathogens, pathogen characteristics, or host characteristics (for example, up to thousands per assay) on multiple patient samples, simultaneously (for example, up to hundreds of thousands per assay) to enable clinical diagnosis. While the present disclosure will be focused on embodiments relating to COVID-19 testing, the methods disclosed herein are applicable to the diagnosis of any infectious diseases, cancer, autoimmune diseases, and other systemic perturbations with molecular alterations.

In one embodiment, the presence of at least one biomolecule in a sample is determined by the following steps: (a) obtaining a sample from a subject; (b) incubating the sample with a composition comprising a first bead, wherein said first bead is conjugated with (i) a first barcode moiety, (ii) a second barcode moiety, and (iii) a ligand capable of binding to the biomolecule, thereby producing a composition comprising a first construct, wherein said first construct comprises the first bead, the ligand and the biomolecule; (c) incubating the composition of (b) with a composition comprising a second bead, wherein said second bead is conjugated with an agent capable of binding to the biomolecule of the first construct, thereby producing a composition comprising a second construct, wherein said second construct comprises the first bead, the ligand, the biomolecule, the agent, and the second bead; (d) washing the composition of (c) under conditions that allow removal of unbound beads; (e) separating the first bead from the second bead under conditions that allow detection of the first bead comprising the first barcode moiety and the second barcode moiety; and (f) determining the presence of the first barcode moicty and the second barcode moiety.

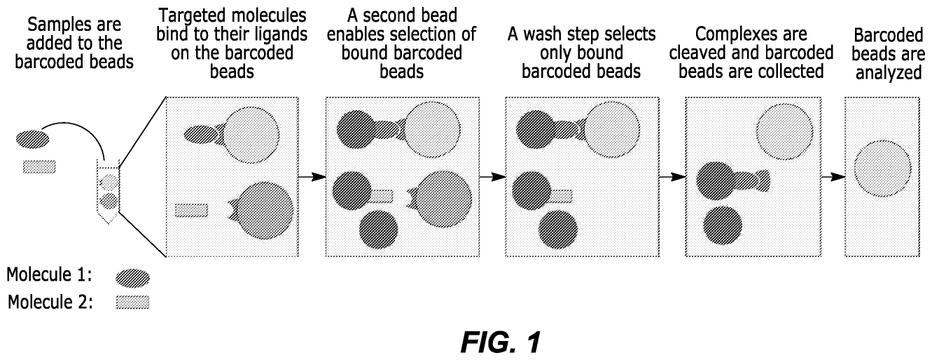

The methods of the present disclosure are based on a two-bead system. In various embodiments, the beads have distinct physical attributes (for example, size and magnetism or a combination of properties) with one type of bead coupled to a barcoding strategy (for example, isotope- or oligonucleotide-based). In various embodiments, the two beads are brought together by a system of biomolecules. In various embodiments, conjugated beads are separated before analysis (for example, by specific proteolytic cleavages, chemical-based disruption, mechanical disruption, or heat-based dehybridization step). In various embodiments, readout methods include, but are not limited to, flow cytometry, mass cytometry, fluorescence microscopy, electron microscopy, isotope-based imaging, and sequencing. Exemplary steps of the disclosed methods are illustrated in FIGS. 1-3, 6 and 7.

As described herein, one application of the present disclosure relates to the detection and/or quantitation of nucleic acid molecules from human patient samples where the patient is infected with a pathogen.

The present disclosure also provides, in various embodiments, methods for determining the presence of an antibody that recognizes a viral pathogen, such as a coronavirus antibody, in a sample. In exemplary embodiments, the method comprises (a) obtaining a blood sample from a human subject; (b) incubating the sample with a composition comprising a first bead, wherein said first bead is conjugated with (i) a first barcode moiety comprising a first isotope or a first combination of isotopes, (ii) a second barcode moiety comprising a second isotope or a second combination of isotopes, and (iii) a ligand comprising a coronavirus spike or nucleocapsid protein or fragment thereof capable of binding to the coronavirus antibody, thereby producing a composition comprising a first construct, wherein said first construct comprises the first bead, the ligand and the coronavirus antibody; (c) incubating the composition of (b) with a composition comprising a second bead, wherein said second bead is conjugated with an agent comprising an anti-human immunoglobulin antibody capable of binding to the coronavirus antibody of the first construct, thereby producing a composition comprising a second construct, wherein said second construct comprises the first bead, the ligand, the coronavirus antibody, the agent, and the second bead; (d) washing the composition of (c) under conditions that allow removal of unbound beads; (c) separating the first bead from the second bead under conditions that allow detection of the first bead comprising the first barcode moiety and the second barcode moiety; and (f) determining the presence of the first barcode moiety and the second barcode moicty.

The present disclosure also provides, in various embodiments, methods for determining the presence of at least one biomolecule in a sample comprising: (a) obtaining a sample from a subject; (b) incubating the sample with a composition comprising a plurality of ligand beads, wherein each ligand bead is conjugated with (i) a first barcode moiety that is unique for each sample, (ii) a second barcode moiety that is unique for each associated ligand, and (iii) a ligand capable of binding to the biomolecule, thereby producing a composition comprising a first construct, wherein said first construct comprises the first bead, the ligand and the biomolecule; (c) incubating the composition of (b) with a composition comprising a plurality of detection beads, wherein each detection bead is conjugated with an agent capable of binding to the biomolecule of the first construct, thereby producing a composition comprising a second construct, wherein said second construct comprises the first bead, the ligand, the biomolecule, the agent, and the second bead; (d) washing the composition of (c) under conditions that allow removal of unbound beads; (e) separating the first bead from the second bead under conditions that allow detection of the first bead comprising the first barcode moiety and the second barcode moiety; and (f) determining the presence of the first barcode moiety and the second barcode moiety.

### Definitions

The plural herein shall equally denote the singular, and the singular shall equally denote the plural wherever reasonable. The words “for example” or “by way of example” or similar phrases shall be interpreted as equivalent to “for example but not by way of limitation”, such that any example shall not limit the generality to which the example pertains.

As used herein, the term “sample” or “biological sample” encompasses a variety of sample types obtained from a variety of sources, which sample types contain biological material. For example, the term includes biological samples obtained from a subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, serum, saliva, nasal/oropharyngeal swab, semen, urine, lymph, cerebrospinal fluid, interstitial fluid, spinal fluid, peritoneal fluid, pleural fluid, amniotic fluid sample, and the like; stool; bile; bone marrow; skin sample (e.g., skin biopsy); and viruses or viral particles obtained from an individual.

In various embodiments, the methods disclosed herein provide for simultaneous testing of one or more patient samples to determine the presence of one or more biomolecule(s). In various embodiments 1, 2, 3, 4, 5, 6, 7, 8, 9, 10, 25, 50, 100, 500, 1000 or more patient samples may be tested simultaneously using the methods disclosed herein.

As used herein, the term “biomolecule” refers to an antibody, polypeptide, a nucleic acid or fragments thereof which is detected in a sample.

As described more fully herein, in various aspects the subject methods may be used to detect a variety of biomolecules from such samples. Biomolecules include, but are not necessarily limited to, nucleic acid (e.g., DNA and/or RNA), protein/peptide/polypeptide, cells (e.g., circulating cells and/or circulating tumor cells), viruses, and many other components that may be present in a biological sample.

The term “nucleic acid” (e.g., as it relates to a biomolecule) refers to a polymer composed of a multiplicity of nucleotide units (ribonucleotide or deoxyribonucleotide or related structural variants) linked via phosphodiester bonds. A nucleic acid (or polynucleotide) can be of substantially any length, typically from about six (6) nucleotides to about 10⁹ nucleotides or larger. Nucleic acids include RNA, cDNA, genomic DNA. In various embodiments, the RNA, cDNA, or genomic DNA is RNA, cDNA, or genomic DNA from a pathogen.

The term “protein” (e.g., as it relates to a biomolecule) refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively.

A typical full-length (intact) immunoglobulin (antibody) structural unit is known to comprise a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments that can be produced, inter alia, by digestion with various peptidases. Thus, for example, pepsin digests an antibody below the disulfide linkages in the hinge region to produce F(ab)′₂: a dimer of Fab which itself is a light chain joined to V_{H}-C_{H}I by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W.E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes whole antibodies, antibody fragments either produced by the modification of whole antibodies or synthesized de novo using recombinant DNA methodologies.

The term “monoclonal antibody” refers to an antibody obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are typically highly specific, being directed against a single epitope. In contrast to polyclonal antibody preparations which typically include different antibodies directed against different determinants (epitopes), each monoclonal antibody is directed against a single determinant on the antigen. The term “monoclonal” indicates the character of the antibody as being obtained from, or one of, a substantially homogeneous population of antibodies, and is not to be construed as requiring production of the antibody by any particular method. For example, monoclonal antibodies may be made by a variety of techniques, including, but not limited to, the hybridoma method (see, c.g., Kohler and Milstein. (1975) Nature, 256:495-497; Hongo et al. (1995) Hybridoma, 14 (3): 253-260; Harlow et al. (1988) Antibodies: A Laboratory Manual (Cold Spring Harbor Laboratory Press, 2d ed.); Hammerling et al. (1981) In: Monoclonal Antibodies and T-Cell Hybridomas 563-681 (Elsevier, N.Y.)), recombinant DNA methods (sec, e.g., U.S. Pat. No. 4,816,567), phage-display technologies (see, e.g., Clackson et al. (1991) Nature, 352: 624-628; Marks et al. (1992) J. Mol. Biol. 222: 581-597; Sidhu et al. (2004) J. Mol. Biol. 338(2): 299-310; Lee et al. (2004) J. Mol. Biol. 340(5): 1073-1093; and the like), and technologies for producing human or human-like antibodies in animals that have parts or all of the human immunoglobulin loci or genes encoding human immunoglobulin sequences (see, e.g., PCT Patent Publication Nos: WO 1998/24893; WO 1996/34096; WO 1996/33735; and WO 1991/10741; U.S. Pat. Nos: 5,545,807; 5,545,806; 5,569,825; 5,625,126; 5,633,425; and 5,661,016; Jakobovits et al. (1993) Nature 362: 255-258; Bruggemann et al. (1993) Year in Immunol. 7: 33; Marks et al. (1992) Bio/Technology 10: 779-783; Lonberg et al. (1994) Nature 368: 856-859; Morrison (1994) Nature 368: 812-813; Fishwild et al. (1996) Nature Biotechnol. 14: 845-851); Neuberger (1996) Nature Biotechnol. 14: 826; Lonberg and Fluszar (1995) Intern. Rev. Immunol. 13: 65-93; and the like).

In various exemplary embodiments, the antibody is a coronavirus antibody is an anti-Spike protein antibody for SARS-COV-1 or SARS-COV-2.

As used herein, the term “subject” (e.g., as it relates to a biomolecule) refers to an animal, plant, fungi, protist, bacterium or archaea for which a sample is obtained from for use in the disclosed methods. Suitable subjects for the methods disclosed herein include animals, e.g., humans, mouse or rat. The subject may be one that exhibits clinical presentations of a disease condition, or has been diagnosed with a disease. In certain aspects, the subject may be one that has been diagnosed with an infection, e.g., COVID-19, or cancer, exhibits clinical presentations of infection or cancer.

As used herein, the term “bead” refers to any type of solid phase particle of any convenient size, of irregular or regular shape, and which is fabricated from any number of known materials such as polystyrene, cellulose, cellulose derivatives, acrylic resins, glass, silica gels, gelatin, polyvinyl pyrrolidone, co-polymers of vinyl and acrylamide, polystyrene cross-linked with divinylbenzene, or the like (as described, e.g., in Merrifield (1964) Biochemistry 3: 1385-1390), polyacrylamides, latex gels, polystyrene, dextran, rubber, silicon, plastics, nitrocellulose, natural sponges, silica gels, controlled pore glass (CPG), metals, cross-linked dextrans (e.g., Sephadex™), agarose gel (Sepharose™), nanoparticle and other solid phase bead supports known to those of skill in the art. In various embodiments, the first or second or both beads is/are magnetic. In various embodiments the methods disclosed herein may comprise multiple beads. In related embodiments, the methods disclosed herein may comprise 2 beads (i.e. a first bead and a second bead). In various embodiments, the methods disclosed herein may comprise 1, 2, 3, 4, 5, 6, 7, 8, 9, 10, 50, 100, 500, 1000 or more beads. As described herein, beads according to various embodiments may be modified to allow conjugation of polymers and/or ligands and/or barcode moieties. By way of example, the beads may be biotinylated or modified to include maleimide-thiol chemistries to allow conjugation of conjugation of polymers and/or ligands and/or barcode moieties.

As used herein, the terms “barcode moiety” or “barcode moieties” are used to refer to a unique combination of molecular marker(s) or tag(s) which is attached to one or more beads. In various embodiments, the barcode moiety is an isotope, oligonucleotide or fluorophore barcode.

In various embodiments, the oligonucleotide barcode moiety is from about 1 to about 100 or more nucleotides in length. In various embodiments, the oligonucleotide barcode moicty is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, or more nucleotides in length. In various embodiments, the oligonucleotide barcode moiety is 6-10 nucleotides in length. In various embodiments, the oligonucleotide barcode is detected by sequencing (See for example, Macosko et al. 2015, Cell 161 (5): 1202-14; Rodriques et al. 2019, Science 363 (6434): 1463-67; Liu et al. 2020, Cell 183 (6): 1665-1681, incorporated by reference in their entirety).

In various embodiments, the oligonucleotide barcode moiety is conjugated to a fluorophore (fluorophore barcode). Various fluorophore which may be used in the fluorophore barcode are known in the art. Fluorophores include, but are not limited to, rhodamines, fluorescein derivatives, Green Fluorescent Protein (GFP), BODIPY® dye, fluorescein, RHODAMINE GREEN™ dye, and OREGON GREEN® dye or derivatives thereof. In various exemplary embodiments, beads containing an oligonucleotide barcode sequences are conjugated by one or more rounds of hybridization-imaging-dehybridization which provide an image with the information of the barcoded beads which can be counted (See for example, co-detection by indexing (CODEX) described in Goltsev et al. 2018, Cell 174 (4): 968-981.e15, which is incorporated by reference in its entirety).

In various embodiments, one or more barcode moieties is associated with a first bead, the second or subsequent bead(s) or all beads. In various aspects, a signal from one or more barcode moieties is used to determine the presence of one or more biomolecule(s). In various embodiments, the presence of a signal from the second barcode moiety is indicative of a current or prior infection from a pathogen associated with the ligand. Examples of barcoded beads and barcoded moieties that may be used in the disclosed methods are known in the art and described in, for example, Dagher et al. 2018, Nature Nanotechnology 13 (10): 925-32; Juncker et al. 2014, Current Opinion in Chemical Biology 18 (February): 29-37; Gold et al. 2010, PloS One 5 (12): e15004 (all of which are incorporated by reference in their entirety). Multiple barcodes can be used on one or more beads as described herein. In some embodiments, one barcode is incorporated or conjugated to a bead in order to allow the identification of the subject (e.g., the subject from where the sample is obtained) where, for example, multiple subjects/samples are processed according to the methods described herein. Moreover, a second barcode moiety is likewise incorporated or conjugated to the bead which serves to identify the ligand that is associated with the bead. Thus, as used herein the phrase “wherein the first barcode moicty identifies the subject” refers to a barcode as described herein that is unique to a specific sample or subject, such that following detection of a biomolecule can be using the beads described herein, the presence of the biomolecule can be attributed to the specific sample or subject. Likewise, the phrase “wherein the second barcode moiety identifies the ligand” refers to a barcode that uniquely identifies the ligand that is associated with the same band.

As used herein, the term “ligand” refers to an antigen or biomolecule-binding fragment of an antigen, biotinylated protein, polymer or a nucleic acid. In various embodiments, the ligand comprises a coronavirus spike or nucleocapsid protein or fragment thereof capable of binding to a coronavirus antibody. In various embodiments, the ligand is associated with a first bead. In various embodiments, the ligand is associated with one or more beads. In various embodiments, the ligand or bead is bioconjugated (for example, biotinylated or Maleimide-thiol reactions) to allow the conjugation of ligands and/or one or more barcode moieties as described herein.

As used herein, the term “agent” or “agents” refers to an antibody, protein or nucleic acid capable of binding to the biomolecule. In various embodiments, the agent is associated with a second bead or subsequent bead(s). In various embodiments, the agent is associated with one or more beads. In various embodiments, the agents capable of binding to the biomolecule of the first construct are antibodies. In related embodiments, the antibodies comprise the same immunoglobulins capable of binding to one or more biomolecules. In certain embodiments, the antibodies comprise different immunoglobulins capable of binding to one or more biomolecules.

In various embodiments, the agents capable of binding to the biomolecule of the first construct are nucleic acids. In related embodiments, the nucleic acids are capable of binding to one or more nucleic acid sequences of the same biomolecule. In certain embodiments, the nucleic acids are capable of binding to one or more nucleic acid sequences of different biomolecules.

As used herein, the term “construct” refers to a mixture of two or more the following one or more bead(s), the ligand, the agent and the biomolecule. In various embodiments, a “first construct” refers to a mixture comprising the first bead, the ligand and the biomolecule. In various embodiments, a “second construct” refers to a mixture of comprising the first bead, the ligand, the biomolecule, the agent, and the second bead.

As used herein, the term “blocking agents” refers to agents used to bind any unbound antibody or protein or nucleic acid on the first bead or second bead or subsequent bead(s). In various embodiments, blocking agents are soluble synthetic proteins or subunits or antigens which block free sites on unbound antibody or protein or nucleic acid.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al. Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

### Methods

The present disclosure provides methods and materials for determining the presence of biomolecules. As described herein, the sample obtained from the subject is incubated, conjugated with or separated from bead compositions. The methods also provide various means conjugating and separating the bead compositions described herein. In various embodiments, bead compositions can be separated using specific proteolytic cleavages, chemical-based disruption, mechanical disruption (e.g. mixing or vortexing), or heat-based dehybridization. Other means of incubating, conjugating and separating beads are known to those of ordinary skill in the art.

In various embodiments, the one or more steps of the disclosed methods may be optionally performed robotically/autonomously (e.g. automated and/or use of robot and/or machine). In exemplary embodiments, the following steps (a) through (f) may be performed robotically/autonomously:

(a) obtaining a sample from a subject; (b) incubating the sample with a composition comprising a plurality of ligand beads, wherein each ligand bead is conjugated with (i) a first barcode moiety that is unique for each sample, (ii) a second barcode moiety that is unique for each associated ligand, and (iii) a ligand capable of binding to the biomolecule, thereby producing a composition comprising a first construct, wherein said first construct comprises the first bead, the ligand and the biomolecule; (c) incubating the composition of (b) with a composition comprising a plurality of detection beads, wherein each detection bead is conjugated with an agent capable of binding to the biomolecule of the first construct, thereby producing a composition comprising a second construct, wherein said second construct comprises the first bead, the ligand, the biomolecule, the agent, and the second bead; (d) washing the composition of (c) under conditions that allow removal of unbound beads; (e) separating the first bead from the second bead under conditions that allow detection of the first bead comprising the first barcode moiety and the second barcode moiety; and (f) determining the presence of the first barcode moiety and the second barcode moicty.

“Detecting” or “determining” as used herein generally means identifying the presence of a biomolecule, such as a nucleic acid or protein or antibody, by the determining the presence one or more barcode moieties. In various embodiments, detection signals are produced by the methods described herein, and such detection signals may be optical signals which may include but are not limited to, colorimetric changes, fluorescence, turbidity, mass-to-charge ratio of ions, and luminescence. Detecting or determining, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments. In various embodiments, the presence one or more barcode moieties is determined using flow cytometry, mass spectrometry, fluorescence microscopy, electron microscopy, isotope-based imaging, Nuclear Magnetic Resonance (NMR), fluorescence resonance energy transfer (FRET), X-ray fluorescence (XRF), or nucleic acid sequencing.

In various embodiments of the methods disclosed herein, the presence of 1, 2, 3, 4, 5, 6, 7, 8, 9, 10, 50, 100, 1000 or more biomolecules are determined.

### Pathogens

In various embodiments, the biomolecule is from a pathogen. In related embodiments the pathogen is a virus, bacterium, fungi, protozoa, or worm.

The present disclosure provides, in various embodiments, methods for detecting the presence of one or more of the following exemplary pathogens:

Exemplary pathogenic viruses or virus particles include, but is not limited to, for example, adenovirus , alphavirus , calicivirus (e.g., a calicivirus capsid antigen), coronavirus polypeptides, distemper virus , Ebola virus polypeptides, enterovirus , flavivirus , hepatitis virus (AE), herpesvirus, infectious peritonitis virus, leukemia virus, marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, rhinoviruses and rotavirus. In certain embodiments, the virus is SARS-COV-1, SARS-COV-2, respiratory syncytial virus (RSV) human immunodeficiency virus (HIV), herpes simplex virus (HSV), human papillomavirus (HPV), influenza or parainfluenza virus.

Exemplary pathogenic bacterium include, but are not limited to, for example, members of the genus Actinomyces, Bacillus, Bacteroides, Bordetella, Bartonella, Borrelia (e.g., B. burgdorferi OspA), Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus polypeptides, Helicobacter, Klebsiella, L-form bacteria, Leptospira, Listeria, Mycobacteria, Mycoplasma, Neisseria, Neorickettsi, Nocardia, Pasteurella, Peptococcus, Peptostreptococcus, Pneumococcus polypeptides (i.e., S. pneumoniac polypeptides), Proteus, Pseudomonas, Rickettsia, Rochalimaca, Salmonella, Shigella, Staphylococcus, group A streptococcus (e.g., S. pyogenes), group B streptococcus (S. agalactiae), Treponema, and Yersinia. In certain embodiments, the pathogenic bacterium is Staphylococcus aureus, E. Coli, Listeria monocytogenes, Yersinia pestis (bubonic plaque), Mycobacterium tuberculosis

Exemplary pathogenic fungus includes, but are not limited to, for example, Aspergillus, Blastomyces, Coccidioides and Pneumocystis. In other related embodiments the fungus is a yeast, which in further embodiments is a Candida, wherein in still further embodiments the Candida is selected from C.albicans, C. glabrata, C. krusci, C. lusitaniac, C. tropicalis and C. parapsilosis. In certain embodiments, the pathogenic fungus/yeast is Candida, tinca corporis (ringworm) Trichophyton, Microsporum, and Epidermophyton.

Exemplary protozoan pathogens include, but are not limited to, for example, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aclurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Sctaria, Spirocerca Spirometra, Stephanofilaria, Strongyloides, Strongylus, Thelazia, Toxascaris, Toxocara, Trichinella, Trichostrongylus, Trichuris, Uncinaria, and Wuchereria, Pneumocystis, Sarcocystis, Schistosoma, Theileria, Toxoplasma, and Trypanosoma. In certain embodiments, the protozoan pathogen is entamoeba histolytica (Amoebiasis) or Plasmodium falciparum (malaria).

Exemplary worm pathogens include, but are not limited to, for example, platyhelminthes or flatworms (flukes and tapeworms) and the nematoda or roundworms.

### Coronaviruses

Coronaviruses (CoV) have repeatedly emerged from wildlife hosts into humans and livestock animals to cause epidemics with significant morbidity and mortality. The emergence of SARS-COV-2, the virus that causes COVID-19 in 2019 and the rapid, global spread of infection in humans highlights the need for developing therapeutics and vaccines to limit coronavirus epidemics (Wu F, et al., 2020, Nature 1-8; Zhou P. et al., 2020, Nature 1-4; and Zhu N, et al., 2020, N Engl J Med NEJMoa2001017).

Coronaviruses (CoVs) are the largest group of viruses belonging to the Nidovirales order, which includes Coronaviridae, Arteriviridae, and Roniviridae families. The Coronavirinae comprise one of two subfamilies in the Coronaviridae family, with the other being the Torovirinae. The Coronavirinae are further subdivided into four groups, the alpha, beta, gamma and delta coronaviruses. The viruses were initially sorted into these groups based on serology but are now divided by phylogenetic clustering.

All viruses in the Nidovirales order are enveloped, non-segmented positive-sense RNA viruses. They all contain very large genomes for RNA viruses, with Coronavirinae having the largest identified RNA genomes, containing approximately 30 kilobase (kb) genomes. Other common features within the Nidovirales order include: i) a highly conserved genomic organization, with a large replicase gene preceding structural and accessory genes; ii) expression of many nonstructural genes by ribosomal frameshifting; iii) several unique or unusual enzymatic activities encoded within the large replicase-transcriptase polyprotein; and iv) expression of downstream genes by synthesis of 3′ nested sub-genomic mRNAs. In fact, the Nidovirales order name is derived from these nested 3′ mRNAs as nido is Latin for “nest”. The major differences within the Nidovirus families are in the number, type, and sizes of the structural proteins. These differences cause significant alterations in the structure and morphology of the nucleocapsids and virions.

Coronaviruses contain a non-segmented, positive-sense RNA genome of ˜30 kb. The genome contains a 5′ cap structure along with a 3′ poly (A) tail, allowing it to act as a mRNA for translation of the replicase polyproteins. The replicase gene encoding the nonstructural proteins (Nsps) occupies two-thirds of the genome, about 20 kb, as opposed to the structural and accessory proteins, which make up only about 10 kb of the viral genome. The 5′ end of the genome contains a leader sequence and untranslated region (UTR) that contains multiple stem loop structures required for RNA replication and transcription. Additionally, at the beginning of each structural or accessory gene are transcriptional regulatory sequences (TRSs) that are required for expression of each of these genes (see section on RNA replication). The 3′UTR also contains RNA structures required for replication and synthesis of viral RNA. The organization of the coronavirus genome is 5′-leader-UTR-replicase—S (Spike)—E (Envelope)—M (Membrane)—N (Nucleocapsid)-3′UTR-poly (A) tail with accessory genes interspersed within the structural genes at the 3′ end of the genome. The accessory proteins are almost exclusively non-essential for replication in tissue culture; however some have been shown to have important roles in viral pathogenesis.

The present disclosure provides, in various embodiments, methods for determining the presence of one or more coronaviruses. Non-limiting examples of coronaviruses include SARS-Related coronaviruses, severe acute respiratory syndrome coronavirus-2. (SARS-COV-2), severe acute respiratory syndrome coronavirus (SARS-COV), Middle East respiratory syndrome coronavirus (MERS-CoV), human coronavirus 229E (HCoV-229E), human coronavirus 0C43 (HCoV-0C43), human coronavirus HKU1 (HCoV-HKU1), and human coronavirus NL63 (HCoV-NL63).

### Detection Methods

In various embodiments, the determining step (f) comprises using flow cytometry. mass spectrometry, fluorescence microscopy, electron microscopy, isotope-based imaging, Nuclear Magnetic Resonance (NMR), fluorescence resonance energy transfer (FRET), X-ray fluorescence (XRF), or nucleic acid sequencing.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

Numerous embodiments of the present disclosure is demonstrated by the following non-limiting Examples.

### Example 1: Detection of Serum Antibodies Against SARS-CoV-2

This Example describes materials and methods for determining the presence of serum antibodies against SARS-COV-2 using mass cytometry as a readout.

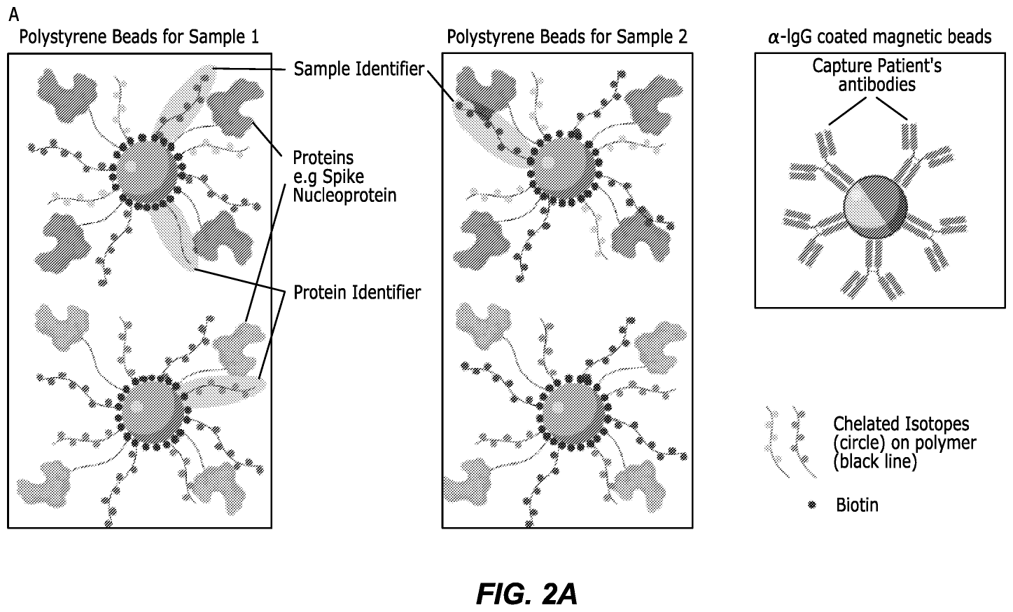

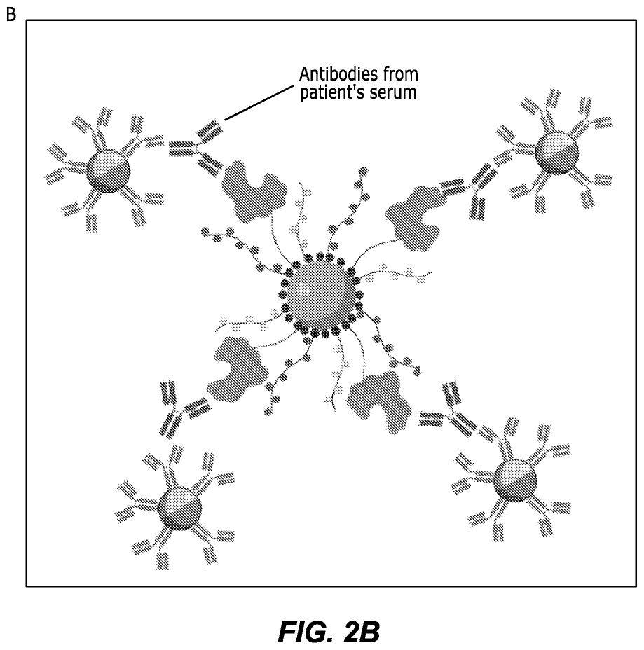

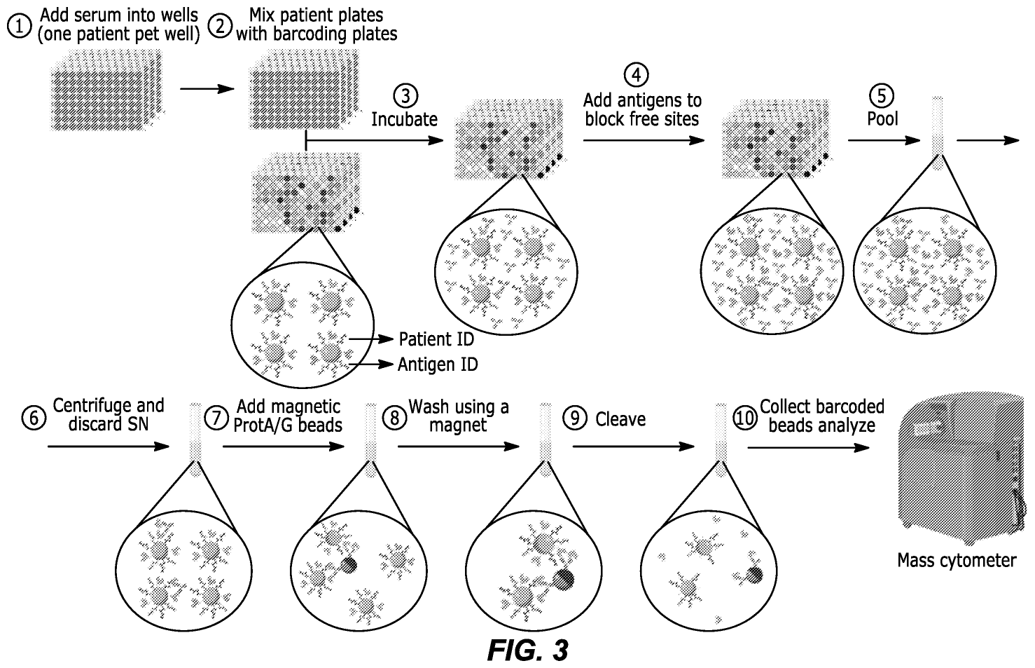

One bead is a polystyrene bead barcoded with a predefined combination of isotopes, and the other is a magnetic bead. The barcoded beads are conjugated to purified viral proteins (e.g., SARS-COV-2 spike, SARS-COV-2 nucleocapsid) or viral protein subunits (FIG. 2A). Two barcodes are present on each polystyrene bead: one for patient sample identification and the other for viral protein or viral protein subunit identification (FIG. 2A). The magnetic beads are conjugated to anti-human immunoglobulins that will bind antibodies (biomolecule) that recognized the proteins or protein fragments conjugated to the barcoded beads (FIG. 2B). The barcoded beads can be isotype (IgG, IgA, IgM) and subclass specific. For SARS-COV-2 serology testing (FIG. 3), serum samples are incubated with the barcoded beads. If a sample contains antibodies against SARS-COV-2, the antibodies will bind to the viral proteins or viral protein subunits. Free sites are blocked with soluble synthetic proteins or subunits, and all samples are pooled and washed to remove any unbound antibodies. The magnetic beads are then added. Polystyrene beads containing bound patient antibodies are linked to the magnetic beads via the anti-human immunoglobulins. Washes are performed on a magnet to retain only barcoded beads from positive samples. Lastly, a cleavage step releases the antigens from the barcoded beads which are then collected and analyzed by mass cytometry.

### Example 2: Two Beads Capture Assay for Antibody Titration of the Spike Protein

This Example describes two bead capture for antibody titration of the Spike protein.

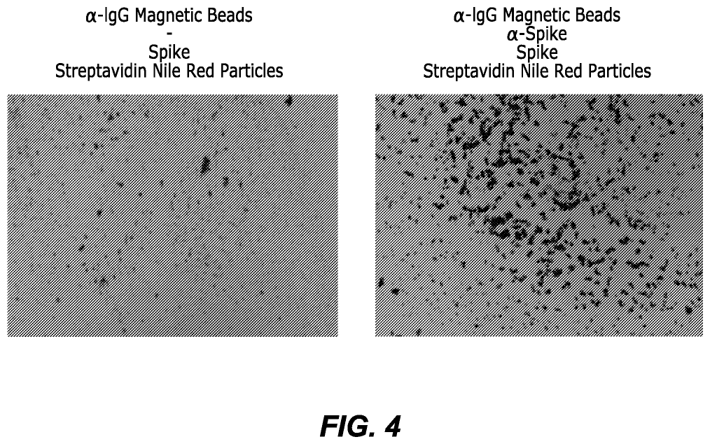

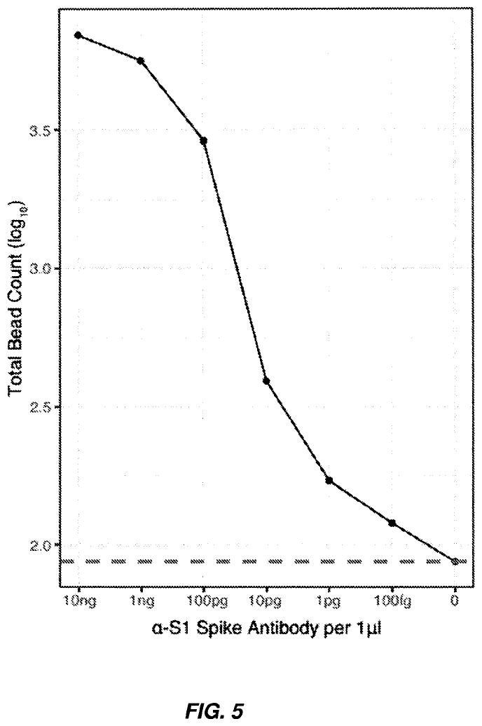

Briefly, 80 μl of streptavidin coated polystyrene Nile red beads (Spherotech) were washed with 500 μl of 0.01% TBS-T (Tris-Buffered pH 7.4, Tween-20) two times (centrifugation at 10,000 rcf). Subsequently, 10 μl of non-biotinylated beads were added and the bead mixer was spun down and resuspended in 80 μl 0.01% TBS-T-PBS. Then 2 μl of biotinylated Spike (Sino Biological) was added and incubated for 45 minutes at room temperature. The mixer was washed two times with cell staining media (CSM, BSA 0.5%, sodium azide 0.01%, PBS) and resuspended in 80 μl CSM. The bead mixer was aliquoted into 8 wells in a 96 well v-bottom plate (10 μl each). Anti-spike antibody was tittered from 0.1× to 10⁶ and 1 μl was added to each well (one well contained two reactions with the 0.1× (one for release and one without release) and the last sample contained no antibody. The mixer was incubated for 30 minutes with agitation. Beads were centrifuged, supernatant discarded and 5 μl of magnetic and IgG beads (RayBiotech) were added to the mixer for 30 minutes at room temperature. 1 μl of the mixer was taken and analyzed by fluorescence microscopy (FIG. 4). Beads were washed 3 times using a magnet and resuspended in CSM. Glycine was added for 10 minutes to disrupt the bond between the antibody and the spike protein to release the polystyrene beads from the magnetic beads. The mixer was pipetted vigorously and then separated by a magnet for 2 minutes. The supernatant (containing the nile red polystyrene beads) was collected to new wells of the 96 well v-bottom plate. A second wash of the magnetic beads with CST was collected and added to their respective wells. Beads were centrifuged and resuspended in 150 μl PBS. 100 μl of each well was acquired using a Cytoflex cytometer (Beckman Coulter). Data was analyzed by quantifying the number of beads identified in the 100 μl of sample (FIG. 5).

### Example 3: Two Beads Capture Assay for Detection of Antibodies Against SARS-COV-2

### Spike S1 in Multiple Human Plasma Samples

This example describes the two bead capture for detection of antibodies against one protein in multiple samples.

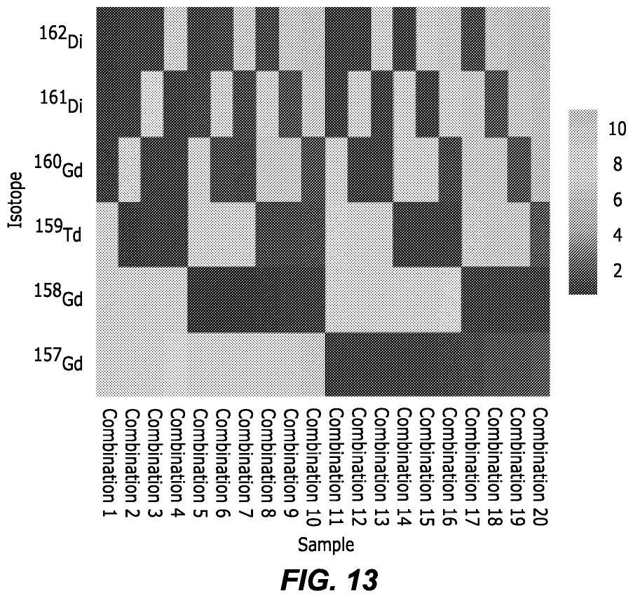

Briefly, 200 μL of 3 μm streptavidin coated polystyrene beads (Spherotech) were washed with 200 μl of 0.001% TBS-T (Tris-Buffered pH 7.4, 0.001% Tween-20) two times (centrifugation at 10,000 rcf). Biotinylated recombinant SARS-COV-2 Spike S1 protein was added (150 ng) and the mixer was incubated for 30 minutes at room temperature. The sample was washed with 1000 μl of 0.001% TBS-T (Tris-Buffered pH 7.4, 0.001% Tween-20) two times (centrifugation at 10,000 ref) and aliquoted into 40 wells in a 96 well v-bottom plate (25 μl each). Each well was incubated with a combination of three of the isotopes between ¹⁵⁹Tb to ¹⁶⁴Dy, and one of the isotopes ¹⁵⁷Gd and ¹⁵⁸Gd, similarly to FIG. 13. Each well was washed with 100 μl of 0.001% TBS-T (Tris-Buffered pH 7.4, 0.001% Tween-20) two times (centrifugation at 10,000 rcf).

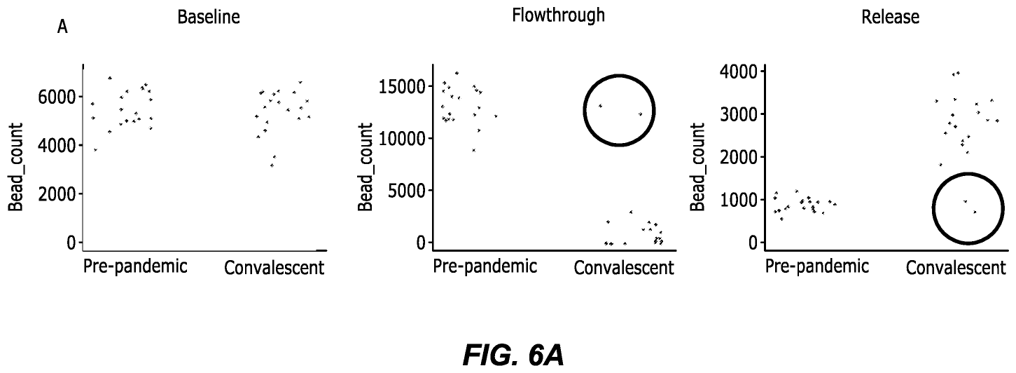

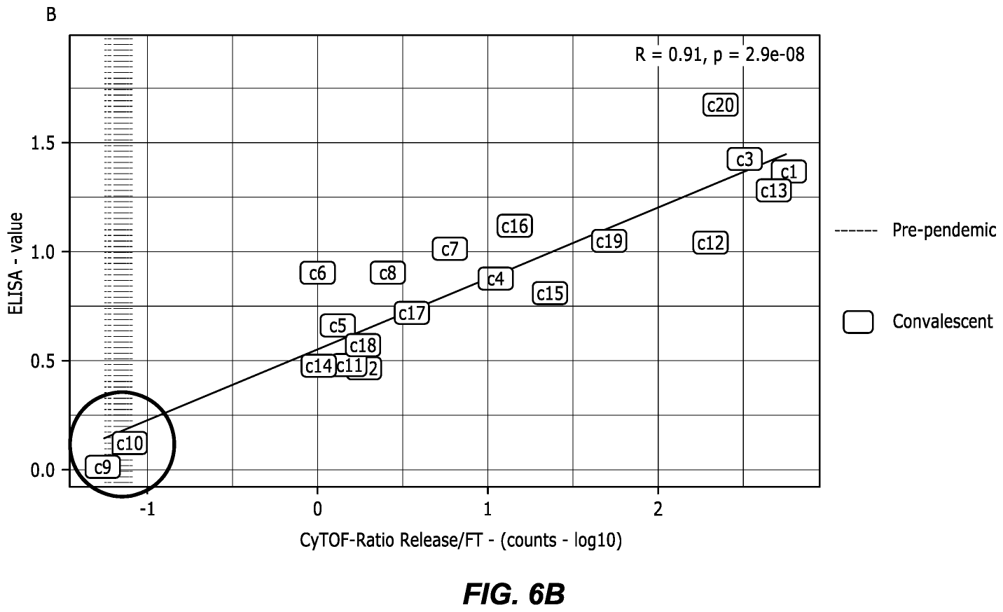

From a cohort of twenty plasma samples obtained from COVID-19 infected individuals (“Convalescent”) and twenty plasma samples from before November 2019 (“Pre-pandemic”), 1 μL of sample was added to each well of the protein-loaded isotope-barcoded beads using the 96-well plate pipettor (Liquidator™, Rainin). The mixer was incubated for 30 minutes at room temperature and washed with 100 μl of 0.001% TBS-T (Tris-Buffered pH 7.4, 0.001% Tween-20) two times (centrifugation at 10,000 rcf) using the 96-well plate pipettor (Liquidator™, Rainin). All wells were pooled and 10% was kept at 4° C. as “Baseline”. The mixture was incubated for 2 hours at room temperature with 450 μl of anti-IgG microbeads (Miltenyi Biotec) and was let to run into a MACS column. The recovered solution was kept at 4° C. as “Flowthrough”. The beads immobilized in the column were washed with a solution of 100 mM glycine pH 2.0 to a tube containing Tris pH 8.0 and kept at 4° C. as “Release”. The “Baseline”, “Flowthrough”, and “Release” samples were then analyzed with CyTOF2 mass cytometer (Fluidigm). The number of beads per barcode was quantified in “Baseline”, “Flowthrough”, and “Release” (FIG. 6A), and the ratio of normalized release value (number of beads in release/number of beads in baseline) to normalized flowthrough value (number of beads in flowthrough/number of beads in baseline) was plotted for each plasma sample to each ELISA value (FIG. 6B).

### Example 4: Two Beads Capture Assay for Detection of Antibodies Against SARS-COV-2

### Spike S1 in Multiple Human Plasma Samples Using Extremely Low Volumes

This example describes the two bead capture for detection of antibodies against one protein in multiple samples diluted to extremely low volumes.

Briefly, 200 μL of 3 μm streptavidin coated polystyrene beads (Spherotech) were washed with 200 μl of 0.001% TBS-T (Tris-Buffered pH 7.4, 0.001% Tween-20) two times (centrifugation at 10,000 rcf). Biotinylated recombinant SARS-COV-2 Spike S1 protein was added (150 ng) and the mixer was incubated for 30 minutes at room temperature. Each well was washed with 100 μl of 0.001% TBS-T (Tris-Buffered pH 7.4, 0.001% Tween-20) two times (centrifugation at 10,000 rcf). Each well was incubated with a combination of three of the isotopes between ¹⁵⁹Tb to ¹⁶⁴Dy, similarly to FIG. 13. Each well was washed with 100 μl of 0.001% TBS-T (Tris-Buffered pH 7.4, 0.001% Tween-20) two times (centrifugation at 10,000 rcf). Protein-loaded isotope-barcoded beads were pooled in one tube containing a final volume of 1000 μl. In parallel, ten 96-well v-bottom plates were prepared contained 44 μL RPMI in each well and kept at 4° C. Twelve solutions containing dilutions of isotopes ¹⁶⁵Ho to ¹⁷⁶Yb were prepared. Using the Tempest (FORMULATRIX®) 1 μl for isotope were aliquoted for combinations of 6 different isotopes to each well, to a total of 924 wells. After the isotope plates and the protein-loaded isotope-barcoded beads mixture was prepared, the mixture of protein-loaded isotope-barcoded beads were dispensed to each of the 924 wells in the isotope-barcoded 96 well v-bottom plate (50 μl each). The mixer was incubated for 2 hours at room temperature and washed with 100 μl of 0.001% TBS-T (Tris-Buffered pH 7.4, 0.001% Tween-20) two times (centrifugation at 10,000 rcf) using the 96-well plate pipettor (Liquidator™, Rainin).

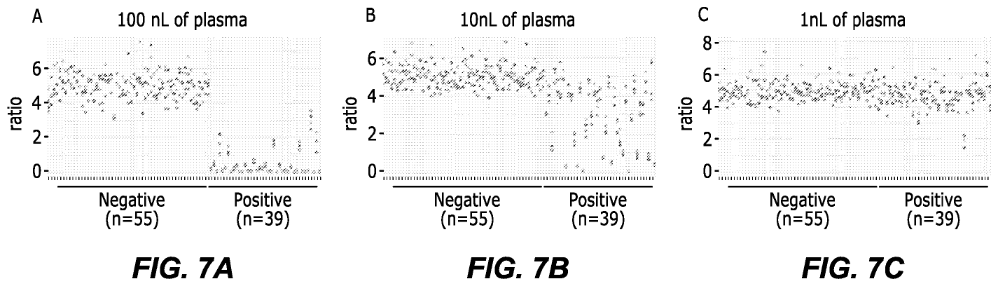

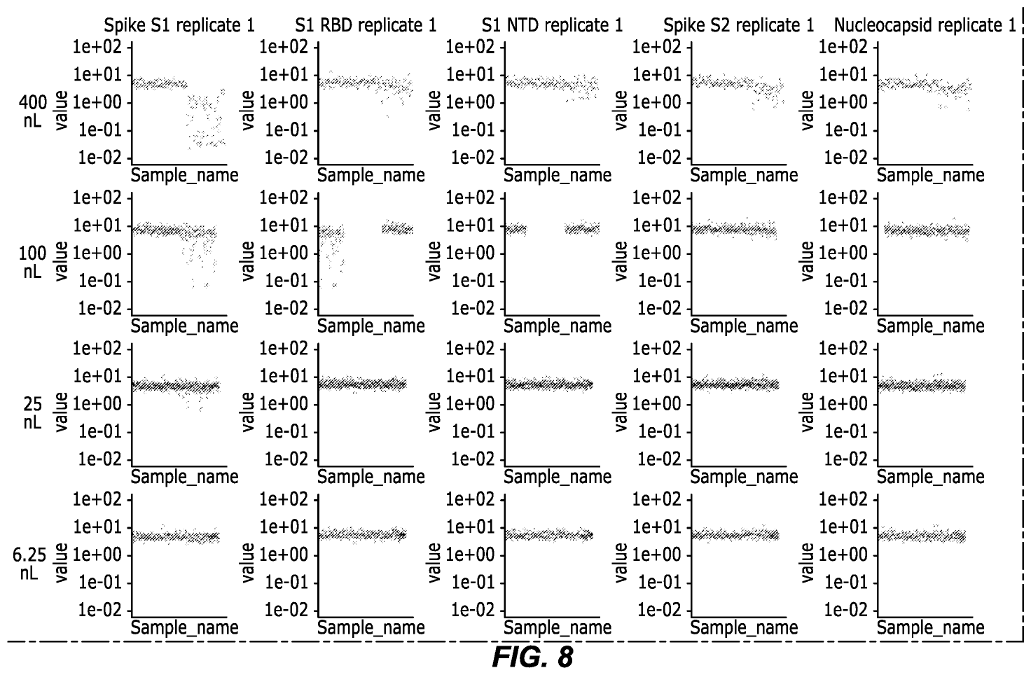

From a cohort of 39 human plasma samples obtained from COVID-19 infected individuals (“Positive”) and 55 human plasma samples from before November 2019 (“Negative”), samples at distinct dilutions (100 nL, 10 nL, and 1 nL) were added to the protein-loaded isotope-barcoded beads using the 96-well plate pipettor (Liquidator™, Rainin). The mixer was incubated for 30 minutes at room temperature and washed with 100 μl of 0.001% TBS-T (Tris-Buffered pH 7.4, 0.001% Tween-20) two times (centrifugation at 10,000 rcf) using the 96-well plate pipettor (Liquidator™, Rainin). All wells were pooled and 10% was kept at 4° C. as “Baseline”. The mixture was incubated for 2 hours at room temperature with 450 μl of anti-IgG microbeads (Miltenyi Biotec) and was let to run into a MACS column. The recovered solution was kept at 4° C. as “Flowthrough”. The “Baseline” and “Flowthrough” samples were then analyzed with CyTOF2 mass cytometer (Fluidigm). The number of beads per barcode was quantified in “Baseline” and “Flowthrough”, and the normalized flowthrough value (number of beads in flowthrough/number of beads in baseline) was plotted for each plasma sample dilution and protein (FIG. 7).

### Example 5: Two Beads Capture Assay for Antibody Detection of Multiple Proteins in Multiple Human Plasma Samples

This example describes the two bead capture for detection of antibodies against multiple proteins in multiple samples. This example describes detection of antibodies against full-length proteins, protein domains, and mutant variants.

Briefly, 1 mL of 3 μm streptavidin coated polystyrene beads (Spherotech) were washed with 1000 μl of 0.001% TBS-T (Tris-Buffered pH 7.4, 0.001% Tween-20) two times (centrifugation at 10.000 rcf). The bead mixer was aliquoted into 20 wells in a 96 well v-bottom plate (50 μl each). Biotinylated recombinant proteins were added to each well (150 ng or less each), as shown.

| | | | | WHO | PANGO | |
|---|---|---|---|---|---|---|
| Bead | Virus | Protein | Domain | label | lineage | WHO label |
| 1 | SARS- | Spike S1 | Full | n/a | n/a | |
| | CoV-2 | | | | | |
| 2 | SARS- | Spike S1 | RBD | n/a | n/a | |
| | CoV-2 | | | | | |
| 3 | SARS- | Spike S1 | NTD | n/a | n/a | |
| | CoV-2 | | | | | |
| 4 | SARS- | Spike S2 | Full | n/a | n/a | |
| | CoV-2 | | | | | |
| 5 | SARS- | Nucleocapsid | Full | n/a | n/a | |
| | CoV-2 | | | | | |
| 6 | SARS- | Spike S1 | Full | In/a | n/a | |
| | CoV-2 | | | | | |
| 7 | SARS- | Spike S1 | RBD | n/a | n/a | |
| | CoV-2 | | | | | |
| 8 | SARS- | Spike S1 | NTD | n/a | n/a | |
| | CoV-2 | | | | | |
| 9 | SARS- | Spike S2 | Full | n/a | n/a | |
| | CoV-2 | | | | | |
| 10 | SARS- | Nucleocapsid | Full | n/a | n/a | |
| | CoV-2 | | | | | |
| 11 | SARS- | Spike S1 | RBD | Gamma | P.1 | E484K, K417T, N501Y |
| | CoV-2 | | | | | |
| 12 | SARS- | Spike S1 | RBD | Alpha | B.1.1.7 | N501Y |
| | CoV-2 | | | | | |
| 13 | SARS- | Spike S1 | RBD | Kappa | B.1.617, | L452R, E484Q |
| | CoV-2 | | | | B.1.617.1, | |
| | | | | | B.1.617.3 | |
| 14 | SARS- | Spike S1 | RBD | Delta | B.1.617.2 | L452R, T478K |
| | CoV-2 | | | | | |
| 15 | SARS- | Spike S1 | | | B.1.617.3 | (T19R, G142D, L452R, E484Q, D614G, P681R |
| | CoV-2 | | | | | |
| 16 | SARS- | Spike S1 | Full | Delta | B.1.617.2 | (157-158) deletion, T19R, G142D, E156G, L452R, |
| | CoV-2 | | | | | T478K, D614G, P681R |
| 17 | SARS- | Spike S1 | Full | Beta | B.1.351 | (LAL242-244) deletion, L18F, D80A, D215G, R246I, |
| | CoV-2 | | | | | K417N, E484K, N501Y, D614G |
| 18 | SARS- | Spike S1 | Full | Alpha | B.1.1.7 | (HV69-70, Y145) deletion, N501Y, A570D, D614G, |
| | CoV-2 | | | | | P681H |
| 19 | | Negative control | | | | |
| 20 | | Negative control | | | | |

The mixer was incubated for 30 minutes at room temperature. Each well was washed with 100 μl of 0.001% TBS-T (Tris-Buffered pH 7.4, 0.001% Tween-20) two times (centrifugation at 10,000 rcf). Each well was incubated with a combination of three of the isotopes between ^{15p}Tb to ¹⁶⁴Dy, similarly to FIG. 13. Each well was washed with 100 μl of 0.001% TBS-T (Tris-Buffered pH 7.4, 0.001% Tween-20) two times (centrifugation at 10,000 rcf). Protein-loaded isotope-barcoded beads were pooled in one tube containing a final volume of 1000 μl. In parallel, ten 96-well v-bottom plates were prepared contained 44 μL RPMI in each well and kept at 4° C. Twelve solutions containing dilutions of isotopes ¹⁶⁵Ho to ¹⁷⁶Yb were prepared. Using the Tempest (FORMULATRIX®) 1 μl for isotope were aliquoted for combinations of 6 different isotopes to each well, to a total of 924 wells.

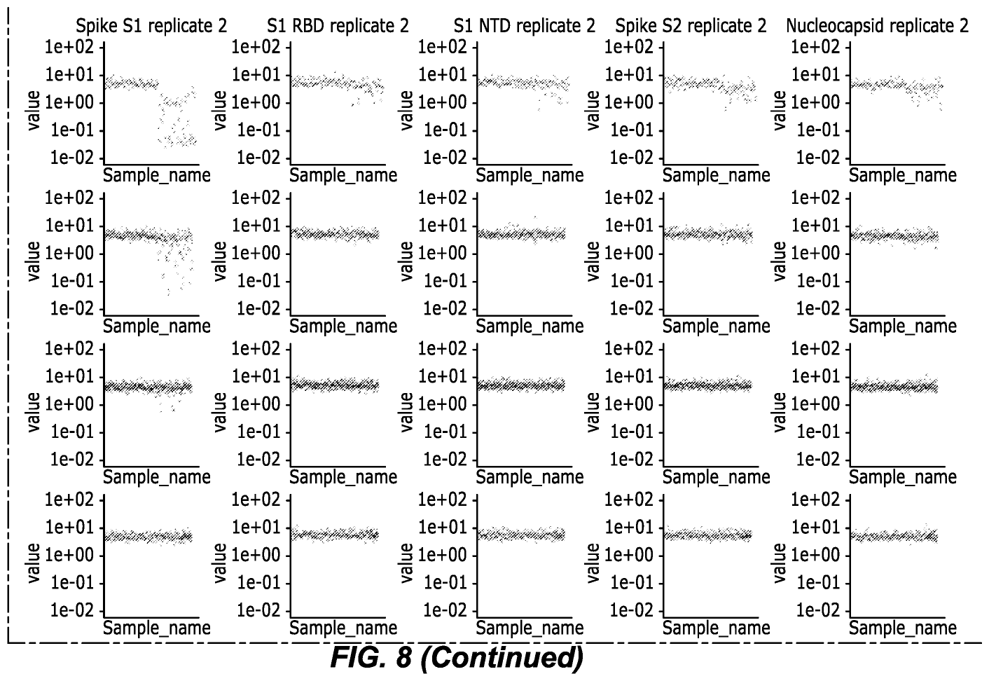

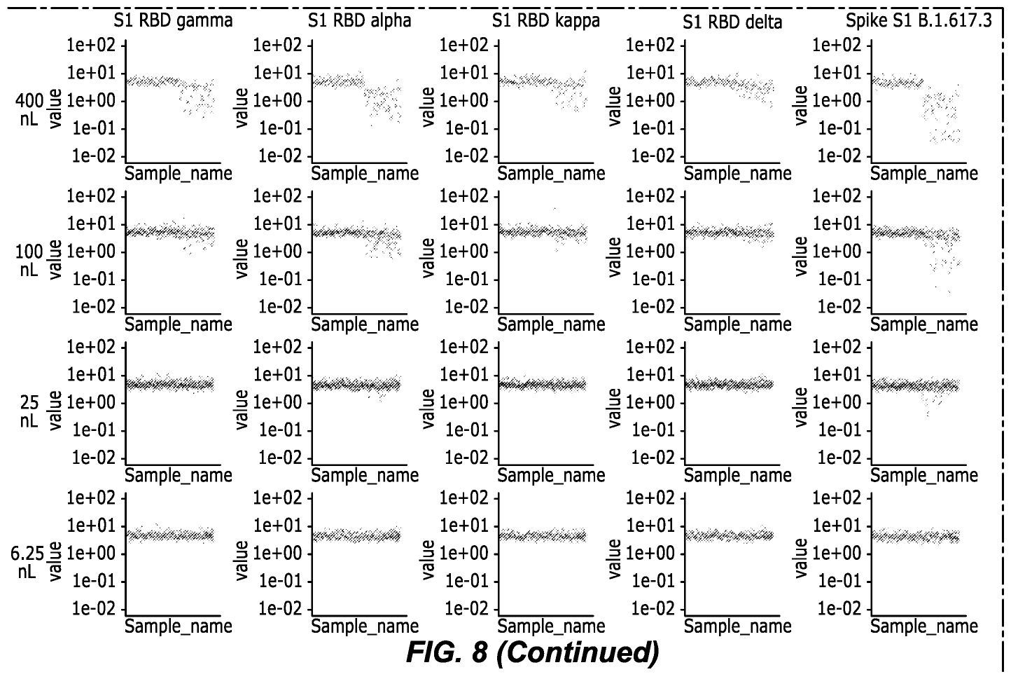

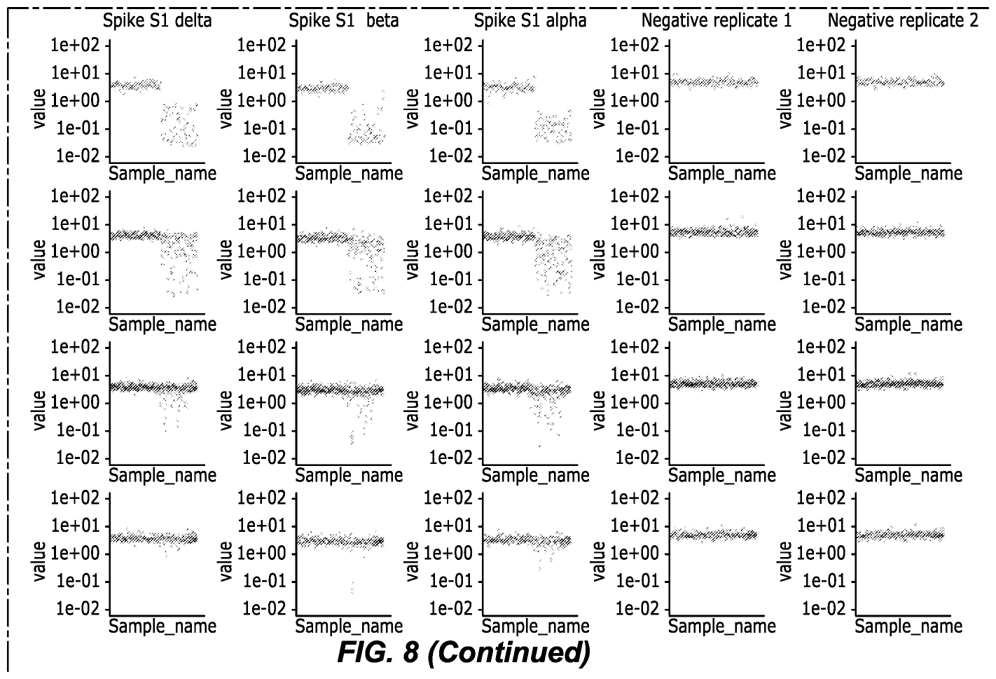

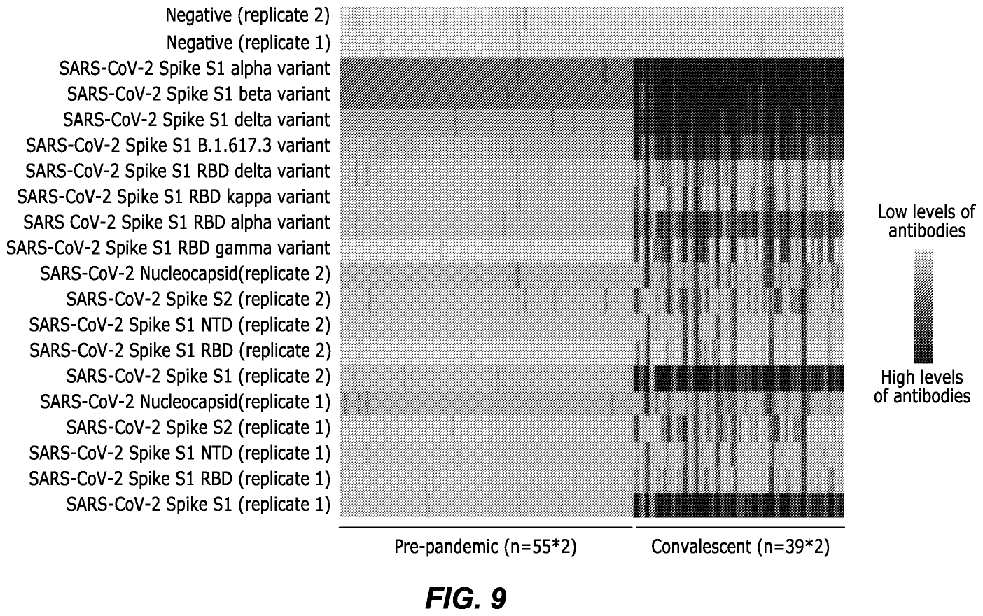

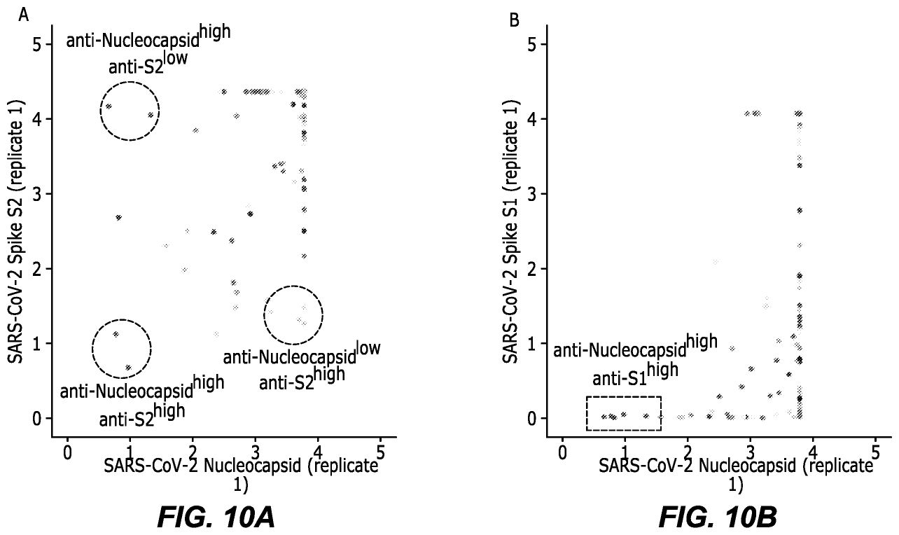

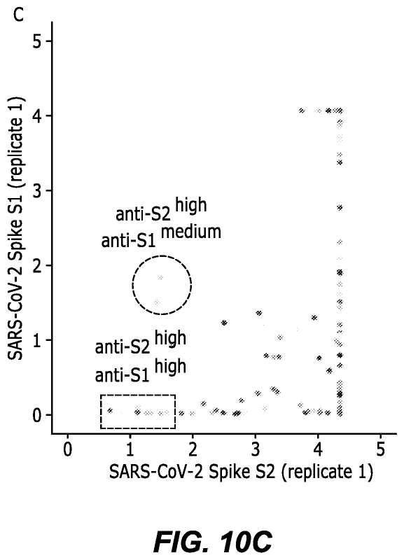

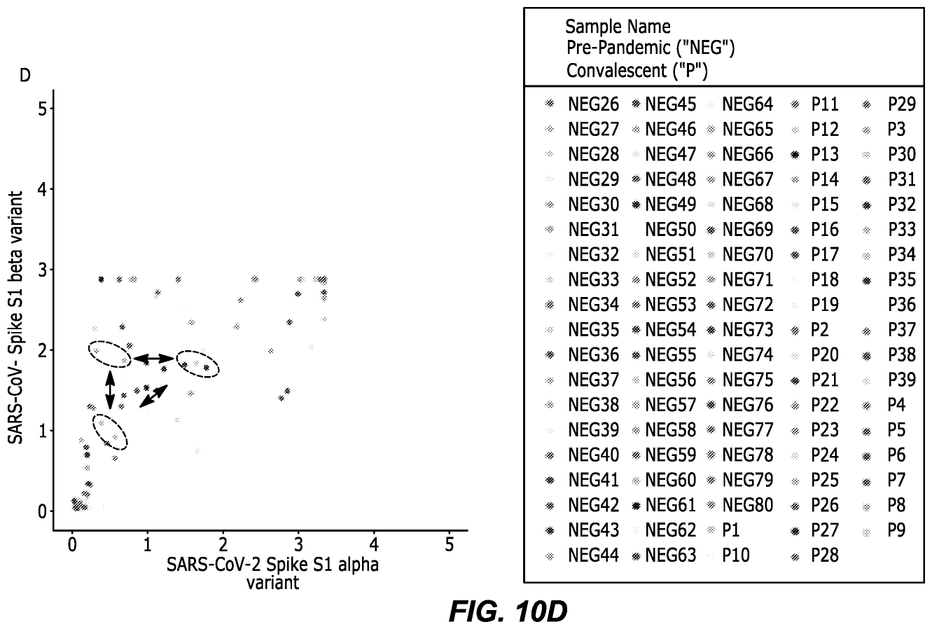

After the isotope plates and the protein-loaded isotope-barcoded beads mixture was prepared, the mixture of protein-loaded isotope-barcoded beads were dispensed to each of the 924 wells in the isotope-barcoded 96 well v-bottom plate (50 μl each). The mixer was incubated for 2 hours at room temperature and washed with 100 μl of 0.001% TBS-T (Tris-Buffered pH 7.4, 0.001% Tween-20) two times (centrifugation at 10,000 rcf) using the 96-well plate pipettor (Liquidator™, Rainin). Human plasma samples pre-aliquoted in 96 well plates at distinct dilutions (400 nL, 100 nL, 25 nL, and 6.25 nL) were added to the protein-loaded isotope-barcoded beads using the 96-well plate pipettor (Liquidator™, Rainin). The mixer was incubated for 30 minutes at room temperature and washed with 100 μl of 0.001% TBS-T (Tris-Buffered pH 7.4, 0.001% Tween-20) two times (centrifugation at 10,000 rcf) using the 96-well plate pipettor (Liquidator™, Rainin). All wells were pooled and 10% was kept at 4° C. as “Baseline”. The mixture was incubated for 2 hours at room temperature with 450 μl of anti-IgG microbeads (Miltenyi Biotec) and was let to run into a MACS column. The recovered solution was kept at 4° C. as “Flowthrough”. The “Baseline” and “Flowthrough” samples were then analyzed with CyTOF2 mass cytometer (Fluidigm). The number of beads per barcode was quantified in “Baseline” and “Flowthrough”, and the normalized flowthrough value (number of beads in flowthrough/number of beads in baseline) was plotted for each plasma sample dilution and protein (FIGS. 8 and 9). Correlation of distinct proteins in each sample shows individual sample characteristics (FIG. 10).

### Example 6: Detection of SARS-COV-2 Viral RNA

This Example describes materials and methods for determining the presence of SARS-CoV-2 viral RNA using mass cytometry as a readout.

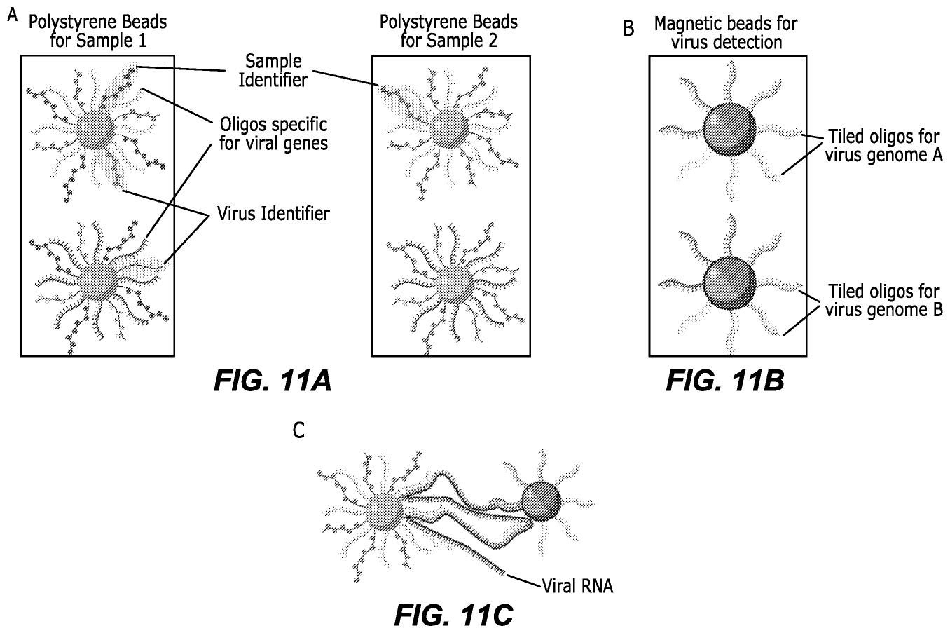

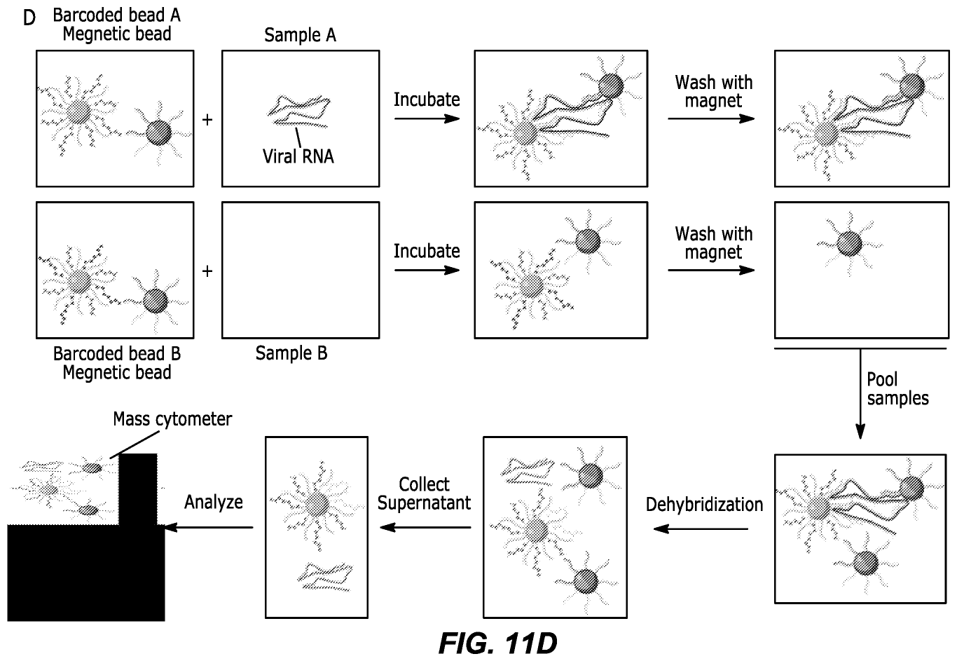

One bead is a polystyrene bead barcoded with a predefined combination of isotopes, and the other is a magnetic bead. Both beads are conjugated to oligonucleotides targeting SARS-CoV-2 RNA. The barcoded beads are conjugated to oligonucleotides targeting specific genes (e.g., S, N, ORFlab genes of SARS-COV-2) (FIG. 11A). Two barcodes are present on each polystyrene bead: one for patient sample identification and the other one for virus gene identification (FIG. 6A). The magnetic beads consist of a mixture of oligonucleotides aimed at substantial coverage of the viral genome (FIG. 11B). For SARS-COV-2 testing, inactivated patient samples are mixed with the two-bead system. If a sample contains SARS-COV-2 RNA, the oligonucleotides on both types of beads hybridize with the viral RNA, linking the two beads (FIG. 11C). If a sample is negative for SARS-COV-2 RNA, no linkage between beads occurs. Using a magnet to hold the beads, washes are performed that retain only barcoded beads from positive samples. The barcoded beads are then collected and analyzed by mass cytometry (FIG. 11D).

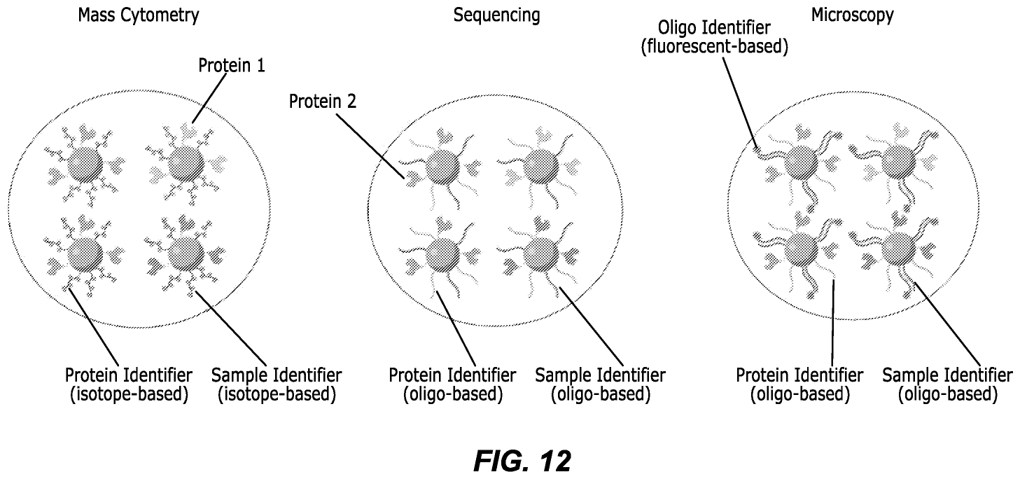

Additionally, generation of barcoded beads with an oligonucleotide strategy enables the use of sequencing and microscopy or mass cytometry as readouts (FIG. 12).

### Example 7: Preparation of Isotope Barcoded Beads and Detection by Mass Spectrometry

This Example describes the preparation of isotope barcoded beads which can be used in the mass spectrometry detection of serum antibodies against SARS-CoV-2 and detection of SARS-CoV-2 viral RNA.

### Preparation of Lanthanide Barcoded Beads

In a 96 well v-bottom plate, 1 μl of 0.5 M lanthanide chloride solutions (Fluidigm) were added in a combinatorial fashion to 25 μl PBS and the volume was brought to 80 μl. Following a 6-choose-3 combinatorial scheme, 20 wells were filled with distinct combinations of 3 of the 6 isotopes. 400 μl of streptavidin coated polystyrene (SP) Nile red beads (Spherotech) were washed with 500 μl of PBS two times (centrifugation at 10,000 rcf) and resuspended in 490 μl of PBS. Then, 10 μl of 100 mM 5 kDa DTPA-PEG-Biotin (Nanocs) were added to the beads and incubated on rotation for 1 hour at room temperature. DTPA-loaded SPs Nile red beads were washed with 500 μl of PBS two times (centrifugation at 10,000 rcf) and resuspended in 500 μl of PBS. Next, 20 μl DTPA-loaded SPs were added to each well containing a distinct combination of lanthanide isotopes and incubated on rotation for 2 hours at room temperature. Lanthanide-loaded SPs Nile red beads were washed with 200 μl of PBS three times (centrifugation at 10,000 rcf), mixed, washed three times with 500 μl of ultrapure water (centrifugation at 10,000 rcf) and resuspended in 500 μl of water. 100 μl of the mixture was acquired using a CyTOF2 mass cytometer (Fluidigm). Data was analyzed by selecting the highest 3 lanthanides on each positive event (FIG. 13).

Mass Cytometry Analysis of Jurkat Cells Stained With Isotopically Labeled Anti-CD45 Following a 12-choose-6 Strategy

Ten 96-well plates were prepared contained 38 μL RPMI in each well and kept at 4° C. 12 solutions containing of 0.015 μg/μL conjugated Anti-CD45 antibody were prepared. Using the Mantis (FORMULATRIX®) we aliquoted 2 μl for antibody for combinations of 6 different antibodies to each well, to a total of 924 wells.

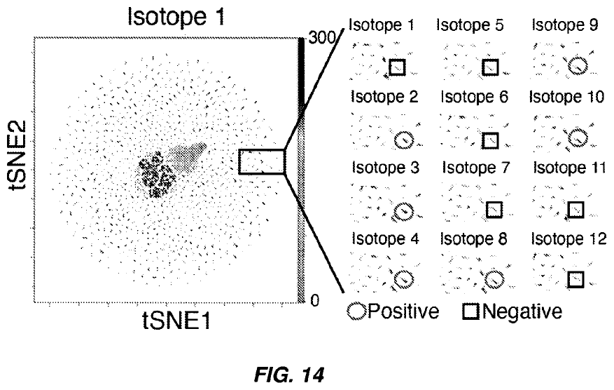

For staining, 10⁵ Jurkat cells (ATCC® TIB-152™) per 50 μl ice-cold RPMI (Thermo Fischer) were prepared. Then, the cells were added on each well using the 96-well plate pipettor (Liquidator™, Rainin) and pipetted 3 times. The cell antibody mixer was incubated for 30 minutes on ice. Plates were subsequently centrifuged and washed twice with 100 μl cold RPMI at 2000 rpm for 5′ at 4° C. Cells were resuspended in 100 μl RPMI and all wells from the 10 plates were collected in two 50 ml tubes. Cells were washed twice with ice-cold RPMI at 2000 rpm for 5′ at 4° C. Cells were then combined in a single 50 ml tube and washed once with ice-cold PBS. The cells were then fixed with ice-cold PBS—1.6% PFA (Thermo Fisher) and incubated for 20 minutes at 4° C. Cells were washed twice ice-cold PBS at 2000 rpm for 5′ at 4° C. Next, the cells were incubated in intercalator solution (1 mL PBS+100 μl 16% PFA+0.22 iridium intercalator) over night at 4° C. The cells were then analyzed with CyTOF2 mass cytometer (Fluidigm) (FIG. 14).

### Example 8: Preparation of Oligo Barcoded Beads and Detection by Sequencing

This example describes the sequencing approach for detecting oligo barcodes from beads.

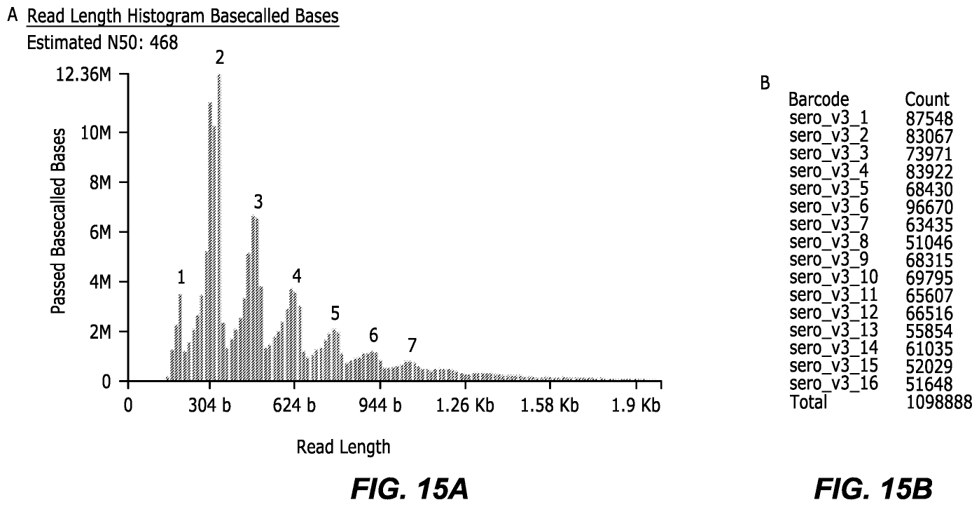

Briefly, 160 μl of 3 μm streptavidin coated polystyrene beads (Spherotech) were washed with 1000 μl of 0.001% TBS-T (Tris-Buffered pH 7.4, 0.001% Tween-20) two times (centrifugation at 10,000 rcf). The bead mixes were aliquoted into 16 wells in a 96 well v-bottom plate (50 μl each). 16 barcoded oligo sequences, which are composed by a linker sequence, restriction enzyme sequence, a variable sequence (barcode) and common sequences, were amplified by biotinylated primers in the 5′ end and quantified using qubit (Thermo Fischer, cat# Q32851). Then the 16 biotinylated barcoded oligoes were individually added to each of the 16 wells containing beads in a concentration of 100 μg per oligo and incubated for 30 min. Each well was washed with 100 μl of 0.001% TBS-T (Tris-Buffered pH 7.4, 0.001% Tween-20) 3 times. All 16 barcodes were then mixed. Next, beads were treated with a restriction enzyme (SmaI (NEB, cat# R0141L) in this example) to digest the oligoes off the beads. The supernatant containing digested DNA was purified from the beads using AMPure beads (Beckman Coulter, cat# A63882). Next DNA was prepared for concatenation using Blunt/TA Ligase Master Mix (NEB, cat# M0367L). The mixture was incubated for 15 minutes at room temperature and the DNA was purified using AMPure beads. The DNA sequencing library was prepare using the Nanopore ligation kit (Nanopore, cat# SQK-LSK109) according to the manufacturer's specifications. The library was sequenced using the MinION flow cells (Nanopore, cat# FLO-MIN106D). The sequenced reads were debarcoded according to the barcode reference sequences and the number of barcodes were quantified (FIG. 15). FIG. 15A, shows the number of concatenated sequences indicated as peaks and size (x-axis). Concatenation of sequences produces ˜3 times more barcoded read counts (˜1 M barcodes, FIG. 15B) compared to the sequenced reads (˜350 k reads).

### Example 9: Two-Bead Capture Assay for Antibody Detection Multiple Human Plasma Samples Using Nanopore Sequencing

This example describes the two-bead capture assay for detection of antibodies against Spike S1 protein in multiple samples using Nanopore sequencing.

Briefly, 160 μl of 3 μm streptavidin coated polystyrene beads (Spherotech) were washed with 1000 μl of 0.001% TBS-T (Tris-Buffered pH 7.4, 0.001% Tween-20) two times (centrifugation at 10,000 rcf). The bead mixes were aliquoted into 16 wells in a 96 well v-bottom plate (50 μl each). 16 barcoded oligo sequences, which are composed by a linker sequence, restriction enzyme sequence, a variable sequence (barcode) and common sequences, were amplified by biotinylated primers in the 5′ end and quantified using qubit (Thermo Fischer, cat# Q32851). Then the 16 biotinylated barcoded oligoes were individually added to each of the 16 wells containing beads in a concentration of 100 μg per oligo and incubated for 30 min. Each well was washed with 100 μl of 0.001% TBS-T (Tris-Buffered pH 7.4, 0.001% Tween-20) 3 times. 16 human plasma samples pre-aliquoted in 96 well plates at a dilution of 400 nL were added to the oligo-loaded barcoded beads using a multichannel pipettor. The mixture was incubated for 30 minutes at room temperature and washed with 100 μl of 0.001% TBS-T (Tris-Buffered pH 7.4, 0.001% Tween-20) two times (centrifugation at 10,000 rcf). Next, all 16 barcodes were pooled. The mixture was incubated for 30 min at room temperature with 160 μl of anti-IgG microbeads (Miltenyi Biotec) and was let to run into a MACS column. The column was washed with 5 ml of 0.001% TBS-T (Tris-Buffered pH 7.4, 0.001% Tween-20). Then the beads were pushed at of the column using a plunger. The enriched barcoded beads bound microbeads were treated with a restriction enzyme (SmaI (NEB, cat# R0141L) in this example) to digest the oligoes off the beads. The supernatant containing digested DNA was purified from the beads using AMPure beads (Beckman Coulter, cat# A63882). The DNA was prepared for concatenation using Blunt/TA Ligase Master Mix (NEB, cat# M0367L). The mixture was incubated for 15 minutes at room temperature and the DNA was purified using AMPure beads. The DNA sequencing library was prepare using the Nanopore ligation kit (Nanopore, cat# SQK-LSK109) according to the manufacturer's specifications. The library was sequenced using the MinION flow cells (Nanopore, cat# FLO-MIN106D). The sequenced reads were debarcoded according to the barcode reference sequences and the number of barcodes were quantified (FIG. 16). FIG. 16, shows on the x-axis the number of reads quantified by Nanopore sequencing, and on the y-axis the median intensity of Spike S1 on each sample using flow cytometry. Nanopore sequencing as a read out is able to detect plasma samples positive for antibodies against Spike S1 protein.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method for determining the presence of at least one biomolecule in a sample comprising:
(a) obtaining a sample from a subject;
(b) incubating the sample with a composition comprising a first bead, wherein said first bead is conjugated with (i) a first barcode moiety, (ii) a second barcode moiety, and (iii) a ligand capable of binding to the biomolecule, thereby producing a composition comprising a first construct, wherein said first construct comprises the first bead, the ligand and the biomolecule;
(c) incubating the composition of (b) with a composition comprising a second bead, wherein said second bead is conjugated with an agent capable of binding to the biomolecule of the first construct, thereby producing a composition comprising a second construct, wherein said second construct comprises the first bead, the ligand, the biomolecule, the agent, and the second bead;
(d) washing the composition of (c) under conditions that allow removal of unbound beads;
(e) separating the first bead from the second bead under conditions that allow detection of the first bead comprising the first barcode moiety and the second barcode moiety; and
(f) determining the presence of the first barcode moiety and the second barcode moiety.

2. The method of claim 1, wherein the subject is an animal, plant, fungi, protist, bacterium or archaea.

3. The method of claim 2, wherein the animal is human.

4. The method of claim 1, wherein the presence of 2, 3, 4, 5 or more biomolecules are determined.

5. The method of claim 1, wherein the sample is a blood, serum, saliva, nasal/oropharyngeal swab, semen, urine, lymph, cerebrospinal fluid, interstitial fluid, spinal fluid, peritoneal fluid, pleural fluid, amniotic fluid, stool, bile, bone marrow, or skin sample.

6. The method of claim 1, wherein the first barcode moiety identifies the subject.

7. The method of claim 1, wherein the second barcode moiety identifies the ligand.

8. The method of claim 1, wherein the biomolecule is an antibody, protein or a nucleic acid.

9. The method of claim 8, wherein the nucleic acid is an RNA.

10. The method of claim 8, wherein the nucleic acid is a DNA.

11. The method of claim 9, wherein the RNA is RNA from a pathogen.

12. (canceled)

13. (canceled)

14. (canceled)

15. (canceled)

16. (canceled)

17. (canceled)

18. The method of claim 1, wherein the agent is an antibody, protein or nucleic acid which binds to the biomolecule.

19. (canceled)

20. (canceled)

21. (canceled)

22. A method for determining the presence of a coronavirus antibody in a sample comprising:
(a) obtaining a blood sample from a human subject;
(b) incubating the sample with a composition comprising a first bead, wherein said first bead is conjugated with (i) a first barcode moiety comprising a first isotope or a first combination of isotopes, (ii) a second barcode moiety comprising a second isotope or a second combination of isotopes, and (iii) a ligand comprising a coronavirus spike or nucleocapsid protein or fragment thereof capable of binding to the coronavirus antibody, thereby producing a composition comprising a first construct, wherein said first construct comprises the first bead, the ligand and the coronavirus antibody;
(c) incubating the composition of (b) with a composition comprising a second bead, wherein said second bead is conjugated with an agent comprising an anti-human immunoglobulin antibody capable of binding to the coronavirus antibody of the first construct, thereby producing a composition comprising a second construct, wherein said second construct comprises the first bead, the ligand, the coronavirus antibody, the agent, and the second bead;
(d) washing the composition of (c) under conditions that allow removal of unbound beads;
(e) separating the first bead from the second bead under conditions that allow detection of the first bead comprising the first barcode moiety and the second barcode moiety; and
(f) determining the presence of the first barcode moiety and the second barcode moiety.

23. The method of claim 22, wherein the coronavirus antibody is an anti-Spike protein antibody for SARS-COV-1 or SARS-COV-2.

24. (canceled)

25. (canceled)

26. (canceled)

27. (canceled)

28. A method for determining the presence of at least one biomolecule in a sample comprising:
(a) obtaining a sample from a subject;
(b) incubating the sample with a composition comprising a plurality of ligand beads, wherein each ligand bead is conjugated with (i) a first barcode moiety that is unique for each sample, (ii) a second barcode moiety that is unique for each associated ligand, and (iii) a ligand capable of binding to the biomolecule, thereby producing a composition comprising a first construct, wherein said first construct comprises the first bead, the ligand and the biomolecule;
(c) incubating the composition of (b) with a composition comprising a plurality of detection beads, wherein each detection bead is conjugated with an agent capable of binding to the biomolecule of the first construct, thereby producing a composition comprising a second construct, wherein said second construct comprises the first bead, the ligand, the biomolecule, the agent, and the second bead;
(d) washing the composition of (c) under conditions that allow removal of unbound beads;
(e) separating the first bead from the second bead under conditions that allow detection of the first bead comprising the first barcode moiety and the second barcode moiety; and
(f) determining the presence of the first barcode moiety and the second barcode moiety.

29. The method of claim 28, wherein the presence of a signal from the second barcode moiety is indicative of a current or prior infection from a pathogen associated with the ligand.

30.-35. (canceled)
